(19) United States
(12) Patent Application Publication
(10) Pub. No.: US 2004/0110758 A1
(43) Pub. Date: Jun. 10, 2004

(54) 4,6-Diaminosubstituted-2-[oxy or aminoxy]-[1,3,5]triazines as protein tyrosine kinase inhibitors

(76) Inventors: Mark R. Player, Phoenixville, PA (US); Nand Baindur, Kendall Park, NJ (US); Benjamin Brandt, Highland Park, NJ (US); Davoud Asgari, Plainsboro, NJ (US); Naresh Chadha, Montville, NJ (US)

Correspondence Address: MORGAN LEWIS & BOCKIUS LLP, 1111 PENNSYLVANIA AVENUE NW, WASHINGTON, DC, 20004, US

(21) Appl. No.: 10/673,521

(22) Filed: Sep. 30, 2003

Related U.S. Application Data
(60) Provisional application No. 60/414,636, filed on Oct. 1, 2002.

(51) Int. Cl. A61K 31/53; C07D 417/02; C07D 413/02; C 07D 4 3/02

(52) U.S. Cl. 514/241; 544/198; 544/60; 544/112; 544/209

(57) ABSTRACT

The invention is directed to compounds of Formulae I, II, III or IV:

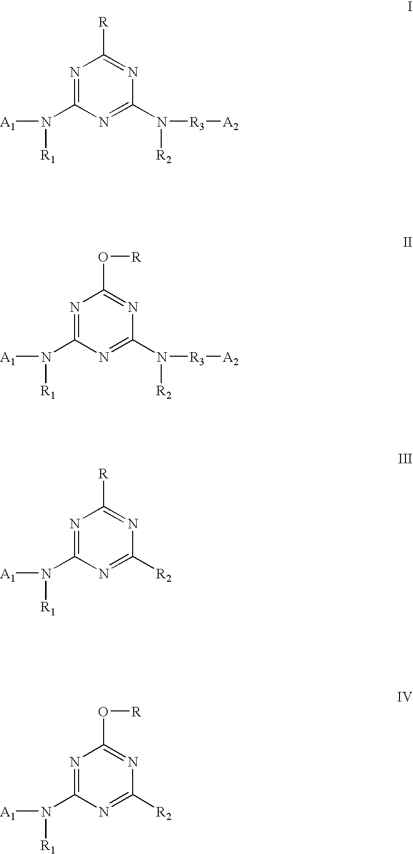

wherein R, R₁, R₂, R₃, A₁ and A₂ are set forth in the specification, as well as solvates, hydrates, tautomers or pharmaceutically acceptable salts thereof, that inhibit protein tyrosine kinases, especially VEGFR-2 (KDR), c-fms, c-met and tie-2 kinases. The invention is also directed toward methods of preparation of the compounds of Formulae I, II, III and IV.

### PRIORITY CLAIM

This application claims benefit under 35 U.S.C. § 119(e) to Provisional Application No. 60/414,636, filed Oct. 1, 2002.

### FIELD OF THE INVENTION

The invention relates to novel substituted triazines that function as protein tyrosine kinase inhibitors. More particularly, the invention relates to 4,6-diaminosubstituted-2-[oxy or aminoxy]-[1,3,5]triazines that function as inhibitors of VEGFR-2 (KDR), c-fms, c-met and tie-2 kinases.

### BACKGROUND OF THE INVENTION

Protein kinases are enzymes that serve as key components of signal transduction pathways by catalyzing the transfer of the terminal phosphate from ATP to the hydroxy group of tyrosine, serine and threonine residues of proteins. As a consequence, protein kinase inhibitors and substrates are valuable tools for assessing the physiological consequences of protein kinase activation. The overexpression or inappropriate expression of normal or mutant protein kinases in mammals has been demonstrated to play significant roles in the development of many diseases, including cancer and diabetes.

Protein kinases can be divided into two classes: those which preferentially phosphorylate tyrosine residues (protein tyrosine kinases) and those which preferentially phosphorylate serine and/or threonine residues (protein serine/threonine kinases). Protein tyrosine kinases perform diverse functions ranging from stimulation of cell growth and differentiation to arrest of cell proliferation. They can be classified as either receptor protein tyrosine kinases or intracellular protein tyrosine kinases. The receptor protein tyrosine kinases, which possess an extracellular ligand binding domain and an intracellular catalytic domain with intrinsic tyrosine kinase activity, are distributed among 20 subfamilies.

Receptor tyrosine kinases of the epidermal growth factor (“EGF”) family, which includes HER-1, HER-2/neu and HER-3 receptors, contain an extracellular binding domain, a transmembrane domain and an intracellular cytoplasmic catalytic domain. Receptor binding leads to the initiation of multiple intracellular tyrosine kinase dependent phosphorylation processes, which ultimately results in oncogene transcription. Breast, colorectal and prostate cancers have been linked to this family of receptors.

Insulin receptor (“IR”) and insulin-like growth factor I receptor (“IGF-1 R”) are structurally and functionally related but exert distinct biological effects. IGF-1R expression has been associated with breast cancer.

Met serves as the high affinity receptor for hepatocyte growth factor (HGF), signalling through which leads to proliferation, scattering and branching morphogenesis. Over-expression of c-Met has been linked to a number of cancers including hereditary papillary renal carcinomas, ovarian cancer, head and neck squamous cell carcinomas and others.

Platelet derived growth factor (“PDGF”) receptors mediate cellular responses that include proliferation, migration and survival and include PDGFR, the stem cell factor receptor (c-kit) and c-fins. These receptors have been linked to diseases such as atherosclerosis, fibrosis and proliferative vitreoretinopathy.

Fibroblast growth factor (“FGR”) receptors consist of four receptors which are responsible for the production of blood vessels, for limb outgrowth, and for the growth and differentiation of numerous cell types.

Vascular endothelial growth factor (“VEGF”), a potent mitogen of endothelial cells, is produced in elevated amounts by many tumors, including ovarian carcinomas. The known receptors for VEGF, flt and KDR, are designated as VEGFR-1 (Flt-1), VEGFR-2 (KDR), VEGFR-3 (Flt-4). A related group of receptors, tie-1 and tie-2 kinases, have been identified in vascular endothelium and hematopoietic cells. VEGF receptors have been linked to vasculogenesis and angiogenesis.

Intracellular protein tyrosine kinases are also known as non-receptor protein tyrosine kinases. Over 24 such kinases have been identified and have been classified into 11 subfamilies. The serine/threonine protein kinases, like the cellular protein tyrosine kinases, are predominantly intracellular.

Diabetes, angiogenesis, psoriasis, restenosis, ocular diseases, schizophrenia, rheumatoid arthritis, cardiovascular disease and cancer are exemplary of pathogenic conditions that have been linked with abnormal protein tyrosine kinase activity. Thus, a need exists for selective and potent small-molecule protein tyrosine kinase inhibitors. U.S. Pat. Nos. 6,383,790; 6,346,625; 6,235,746; 6,100,254 and PCT International Applications WO 01/47897 and WO 01/47921 are indicative of recent attempts to synthesize such inhibitors.

### SUMMARY OF THE INVENTION

The invention answers the current need for selective and potent protein tyrosine kinase inhibitors. One embodiment of the invention is directed to the novel compounds of Formula I:

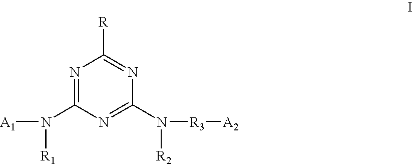

or a solvate, hydrate, tautomer or pharmaceutically acceptable salt thereof, wherein

R is

—OH or —NHORₐ, wherein Rₐ is hydrogen, alkyl, cycloalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORₐ, —COORₐ, —CONRₐR_{b}, —NHCORₐR_{b}, —NHSO₂Rₐ, —SO₂Rₐ, —SO₃Rₐ or —SO₂NRₐR_{b}, wherein Rₐ and R_{b} are independently hydrogen, alkyl, cycloalkyl, aryl or aralkyl;

R₁ is

hydrogen, alkyl, hydroxy or alkoxy;

R₂ is

hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;

R₃ is

a direct link or

C₁₋₆ alkyl, C₁₋₆ alkoxy, C₁₋₆ thioalkyl, C₁₋₆ hydroxyalkyl or C₁₋₆ carboxyalkyl; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{c}, —COOR_{c}, —CONR_{c}R_{d}, —N(R₁)COR_{c}, —SO₂R_{c}, —SO₃R_{c} or —SO₂NR_{c}R_{d};

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —COR_{c}, —COOR_{c}, —CONR_{c} R_{d}, —NHCOR_{c}R_{d}, NHSO₂R_{c}, —SO₂R_{c}, —SO₃R_{c} or —SO₂NR_{c}R_{d}—; or

—COR_{c}, —COOR_{c} or —CONR_{c}R_{d}, wherein

R_{c}, and R_{d} are independently hydrogen, alkyl, cycloalkyl, aryl, aralkyl, heteroaralkyl or heteroaryl.

In another embodiment, the invention is directed to the novel compounds of Formula II:

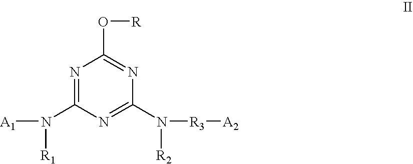

or a solvate, hydrate, tautomer or pharmaceutically acceptable salt thereof, wherein

R is

—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c} or —SO₂NR_{c}R_{d}, wherein R_{c} and R_{d} are independently hydrogen, alkyl, cycloalkyl, aryl or aralkyl;

R₁ is

hydrogen, alkyl, hydroxy or alkoxy;

R₂ is

hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;

R₃ is

a direct link or

C₁₋₆ alkyl, C₁₋₆ alkoxy, C₁₋₆ thioalkyl, C₁₋₆ hydroxyalkyl or C₁₋₆ carboxyalkyl; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂Rₑ, —SO₃Rₑ or —SO₂NRₑR_{f};

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COORₑ, —CONRₑR_{f}, —NHCORₑR_{f}, NHSO₂Rₐ, —SO₂Rₐ, —SO₃Rₐ or —SO₂NRₐR_{b}; or

—CORₑ, —COORₑ or —CONRₑR_{f}, wherein

Rₑ and R_{f} are independently hydrogen, alkyl, cycloalkyl, aryl, aralkyl, heteroaralkyl or heteroaryl.

In yet another embodiment, the invention is directed to the novel compounds of Formula III:

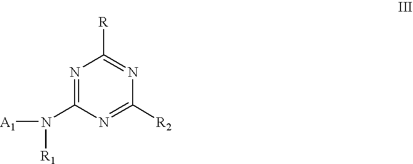

or a solvate, hydrate, tautomer or pharmaceutically acceptable salt thereof, wherein

R is

—OH or —NHORₐ, wherein Rₐ is hydrogen, alkyl, cycloalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORₐ, —COORₐ, —CONRₐR_{b}, —NHCORₐR_{b}, —NHSO₂Rₐ, —SO₂Rₐ, SO₃Rₐ or —SO₂NRₐR_{b}, wherein Rₐ and R_{b} are independently hydrogen, alkyl, cycloalkyl, aryl or aralkyl;

R₁ is

hydrogen, alkyl, hydroxy or alkoxy; and

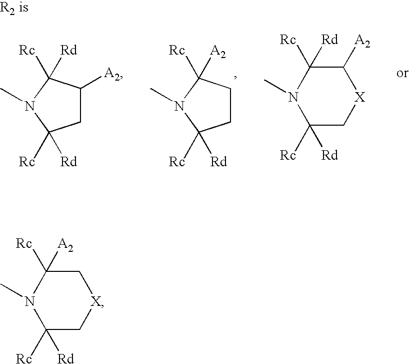

wherein

R_{c} and R_{d} are independently hydrogen or alkyl;

X is N, O or S; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂Rₑ, —SO₃Rₑ or —SO₂NRₑR_{f}; or

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COORₑ, —CONRₑR_{f}, —NHCORₑR_{f}, NHSO₂Rₑ, —SO₂Rₑ, —SO₃Rₑ, or —SO₂NRₑR_{f}, wherein

Rₑ and R_{f} are independently hydrogen, alkyl, cycloalkyl, aryl, aralkyl, heteroaralkyl or heteroaryl.

Yet another embodiment of the invention is directed to the compounds of Formula IV:

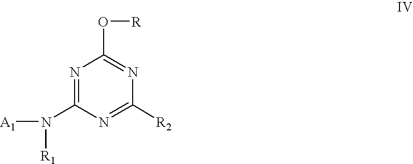

or a solvate, hydrate, tautomer or pharmaceutically acceptable salt thereof, wherein

R is

—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c} or —SO₂NR_{c}R_{d}, wherein R_{c} and R_{d} are independently hydrogen, alkyl, cycloalkyl, aryl or aralkyl;

R₁ is

hydrogen, alkyl, hydroxy or alkoxy; and

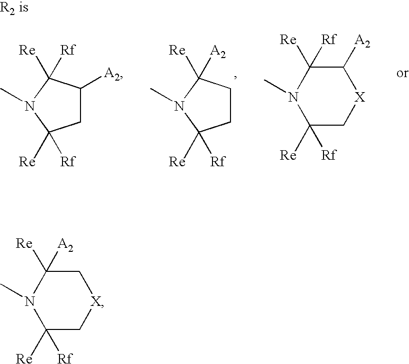

wherein

Rₑ and R_{f} are independently hydrogen or alkyl;

X is N, O or S; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{g}, —COOR_{g}, —CONR_{g}Rₕ, —N(R₁)COR_{g}, —SO₂R_{g}, —SO₃R_{g} or —SO₂NR_{g}Rₕ; or

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —COR_{g}, —COOR_{g}, —CONR_{g}Rₕ, —NHCOR_{g}Rₕ,

NHSO₂R_{g}, —SO₂R_{g}, —SO₃ R_{g} or —SO₂NR_{g}Rₕ, wherein

R_{g} and Rₕ are independently hydrogen, alkyl, cycloalkyl, aryl, aralkyl, heteroaralkyl or heteroaryl.

The compounds of Formulae I and III are especially potent inhibitors of VEGFR-2 (KDR), c-fms, c-met and tie-2 protein tyrosine kinases. The compounds of Formulae II and IV are expected to exhibit similar inhibitory potencies.

In a further embodiment, the invention relates to methods of preparing the compounds of Formulae I, II, III and IV.

The invention also relates to methods of inhibiting protein tyrosine kinase activity in a mammal by administration of a therapeutically effective amount of at least one compound of Formulae I, II, III or IV.

### DETAILED DESCRIPTION OF THE INVENTION

The invention is directed to the novel compounds of Formula I:

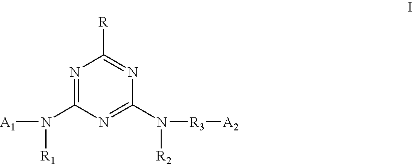

or a solvate, hydrate, tautomer or pharmaceutically acceptable salt thereof, wherein

R is

—OH or —NHORₐ, wherein Rₐ is hydrogen, alkyl, cycloalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORₐ, —COORₐ, —CONRₐR_{b}, —NHCORₐR_{b}, —NHSO₂Rₐ, —SO₂Rₐ, —SO₃Rₐor —SO₂NRₐR_{b}, wherein Rₐ and R_{b} are independently hydrogen, alkyl, cycloalkyl, aryl or aralkyl;

R₁ is

hydrogen, alkyl, hydroxy or alkoxy;

R₂ is

hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;

R₃ is

a direct link or

C₁₋₆ alkyl, C₁₋₆ alkoxy, C₁₋₆ thioalkyl, C₁₋₆ hydroxyalkyl or C₁₋₆ carboxyalkyl; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{c}, —COOR_{c}, —CONR_{c}R_{d}, —N(R₁)COR_{c}, —SO₂R_{c}, —SO₃R_{c} or —SO₂NR_{c} R_{d};

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —COR_{c}, —COOR_{c}, —CONR_{c}R_{d}, —NHCOR_{c}R_{d}, NHSO₂R_{c}, —SO₂R_{c}, —SO₃R_{c} or —SO₂NR_{c}R_{d}; or

—COR_{c}, —COOR_{c} or —CONR_{c}R_{d}, wherein

R_{c} and R_{d} are independently hydrogen, alkyl, cycloalkyl, aryl, aralkyl, heteroaralkyl or heteroaryl.

In another embodiment, the invention is directed to the novel compounds of Formula II:

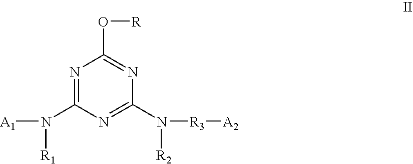

or a solvate, hydrate, tautomer or pharmaceutically acceptable salt thereof, wherein

R is

—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c} or —SO₂NR_{c}R_{d}, wherein R_{c} and R_{d} are independently hydrogen, alkyl, cycloalkyl, aryl or aralkyl;

R₁ is

hydrogen, alkyl, hydroxy or alkoxy;

R₂ is

hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;

R₃ is

a direct link or

C₁₋₆ alkyl, C₁₋₆ alkoxy, C₁₋₆ thioalkyl, C₁₋₆ hydroxyalkyl or C₁₋₆ carboxyalkyl; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂Rₑ—SO₃Rₑ or —SO₂NRₑR_{f};

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COORₑ, —CONRₑR_{f}, —NHCORₑR_{f}, NHSO₂Rₐ, —SO₂Rₐ, —SO₃Rₐ or —SO₂NRₐR_{b}; or

—CORₑ, —COORₑ or —CONRₑR_{f}, wherein

Rₑ and R_{f} are independently hydrogen, alkyl, cycloalkyl, aryl, aralkyl, heteroaralkyl or heteroaryl.

In yet another embodiment, the invention is directed to the novel compounds of Formula III:

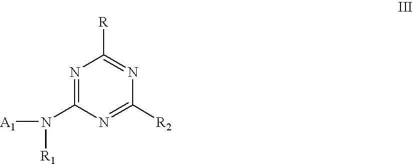

or a solvate, hydrate, tautomer or pharmaceutically acceptable salt thereof, wherein

R is

—OH or —NHORₐ, wherein Rₐ is hydrogen, alkyl, cycloalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORₐ, —COORₐ, —CONRₐR_{b}, —NHCORₐR_{b}, —NHSO₂Rₐ, —SO₂Rₐ, —SO₃Rₐ or —SO₂NRₐR_{b}, wherein Rₐ and R_{b} are independently hydrogen, alkyl, cycloalkyl, aryl or aralkyl;

R₁ is

hydrogen, alkyl, hydroxy or alkoxy; and

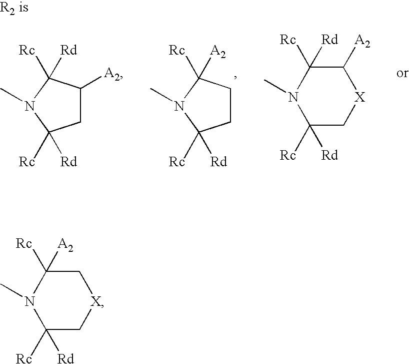

wherein

_{c} and R_{d} are independently hydrogen or alkyl;

X is N, O or S; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂Rₑ, —SO₃Rₑ or —SO₂NRₑR_{f}; or

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COORₑ, —CONRₑR_{f}, —NHCORₑR_{f}, NHSO₂Rₑ, —SO₂Rₑ, —SO₃Rₑ or —SO₂NRₑR_{f}, wherein

Rₑ and R_{f} are independently hydrogen, alkyl, cycloalkyl, aryl, aralkyl, heteroaralkyl or heteroaryl.

Yet another embodiment of the invention is directed to the compounds of Formula IV:

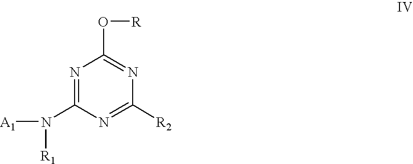

or a solvate, hydrate, tautomer or pharmaceutically acceptable salt thereof, wherein

R is

—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;

A₁ is

a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c} or —SO₂NR_{c}R_{d}, wherein R_{c} and R_{d} are independently hydrogen, alkyl, cycloalkyl, aryl or aralkyl;

R₁ is

hydrogen, alkyl, hydroxy or alkoxy; and

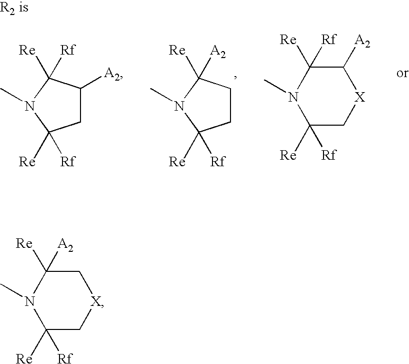

wherein

Rₑ and R_{f} are independently hydrogen or alkyl;

X is N, O or S; and

A₂ is

phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{g}, —COOR_{g}, —CONR_{g}Rₕ, —N(R₁)COR_{g}, —SO₂R_{g}, —SO₃R_{g} or —SO₂NR_{g}Rₕ; or

a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —COR_{g}, —COOR_{g}, —CONR_{g}Rₕ, —NHCOR_{g}Rₕ, NHSO₂R_{g}, —SO₂R_{g}, —SO₃R_{g} or —SO₂NR_{g}Rₕ, wherein

R_{g} and Rₕ are independently hydrogen, alkyl, cycloalkyl, aryl, aralkyl, heteroaralkyl

or heteroaryl.

Preferred compounds of Formula I are those wherein

A₁ is

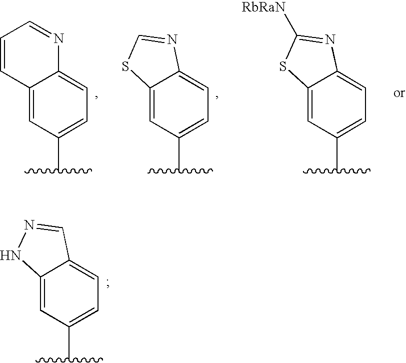

wherein Rₐ and R_{b} are independently —H, —C₁₋₆ alkyl, —CO₂-alkyl, —CO₂—CH₂CH₂NH₂, —CO—(CH₂)₁₄—CO₂H or —(CH₂)₁₄—CO₂H;

R₁ is —H;

R₂ is

—H, -Me, -Et,

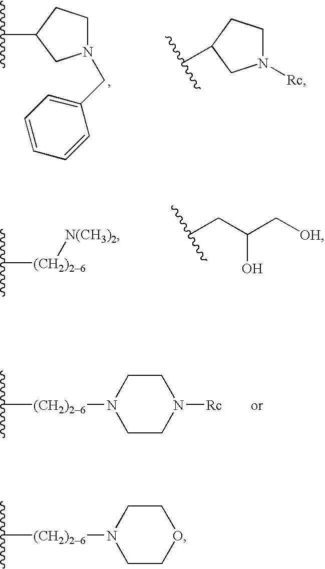

wherein R_{c} is alkyl;

R₃ is

—CH₂—, —CH₂CH₂—, —CH(CH₃)—, —C(CH₃)₂—, —CH(CH₂OH)— or —CH(CH₂CH₂COOH)—; and

A₂ is

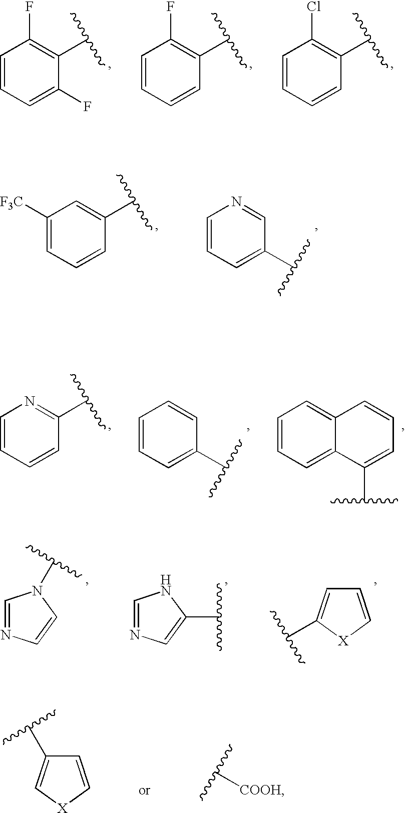

wherein X is O or S.

Particularly preferred compounds of Formula I include, but are not limited to, 4-(Benzothiazol-6-ylamino)-6-(ethyl-benzylamino)-[1,3,5]triazin-2-ol; 4-(Benzothiazol-6-ylamino)-6-(methyl-benzylamino)-[1,3,5]triazin-2-ol; 4-(Benzothiazol-6-ylamino)-6-(benzylamino)-[1,3,5]triazin-2-ol; (R)-4-(Benzothiazol-6-ylamino)-6-(1-phenylethylamino)-[1,3,5]triazin-2-ol; (S)-4-(Benzothiazol-6-ylamino)-6-(1-phenylethylamino)-[1,3,5]triazin-2-ol; (R)-4-(Benzothiazol-6-ylamino)-6-(methyl-1-phenylethylamino)-[1,3,5]triazin-2-ol; (S)-4-(Benzothiazol-6-ylamino)-6-(methyl-1-phenylethylamino)-[1,3,5]triazin-2-ol; (R)-4-(Benzothiazol-6-ylamino)-6-(ethyl-1-phenylethylamino)-[1,3,5]triazin-2-ol; (S)-4-(Benzothiazol-6-ylamino)-6-(ethyl-1-phenylethylamino)-[1,3,5]triazin-2-ol; 4-(Benzothiazol-6-ylamino)-6-(1-methyl-1-phenylethylamino)-[1,3,5]triazin-2-ol; 4-(Benzothiazol-6-ylamino)-6-(2-phenylethylamino)-[1,3,5]triazin-2-ol; 4-(Benzothiazol-6-ylamino)-6-(methyl-2-phenylethylamino)-[1,3,5]triazin-2-ol; 4-(Benzothiazol-6-ylamino)-6-(ethyl-2-phenylethylamino)-[1,3,5]triazin-2-ol; 4-(Benzothiazol-6-ylamino)-6-(2-chloro-benzylamino)-[1,3,5]triazin-2-ol; 4-(Benzothiazol-6-ylamino)-6-(2-fluoro-benzylamino)-[1,3,5]triazin-2-ol; 4-(Benzothiazol-6-ylamino)-6-[(pyridin-3-ylmethyl)-amino)-[1,3,5]triazin-2-ol; 4-(Benzothiazol-6-ylamino)-6-(2,6-difluoro-benzylamino)-[1,3,5]triazin-2-ol; 4-(Benzothiazol-6-ylamino)-6-[methyl-(2-pyridin-2-yl-ethyl)amino]-[1,3,5]triazin-2-ol; 4-(Benzothiazol-6-ylamino)-6-[pyridin-2-ylmethyl)-amino]-[1,3,5]triazin-2-ol; 4-(Benzothiazol-6-ylamino)-6-[benzyl-(1-benzyl-pyrrolidin-3-yl)-amino]-[1,3,5]triazin-2-ol; 4-(Benzothiazol-6-ylamino)-6-(3-fluoro-benzylamino)-[1,3,5]triazin-2-ol; 4-(Benzothiazol-6-ylamino)-6-(2-chloro-6-methyl-benzylamino)-[1,3,5]triazin-2-ol; 4-(Benzothiazol-6-ylamino)-6-(N′-methyl-N′-phenyl-hydrazino)-[1,3,5]triazin-2-ol; 4-(benzothiazol-6-ylamino)-6-[(pyridin-4-ylmethyl)-amino]-[1,3,5]triazin-2-ol; 4-Benzothiazol-6-ylamino)-6-(2-pyridin-3-yl-ethylamino)-[1,3,5]triazin-2-ol; 4-Benzothiazol-6-ylamino)-6-(1-phenyl-propylamino)-[1,3,5]triazin-2-ol; 4-Benzothiazol-6-ylamino)-6-(2-pyridin-2-yl-ethylamino)-[1,3,5]triazin-2-ol; 4-(Benzothiazol-6-ylamino)-6-(1-naphthalen-1-yl-ethylamino)-[1,3,5]triazin-2-ol; 4-(Benzothiazol-6-ylamino)-6-(3-hydroxymethyl-phenylamino)-[1,3,5]triazin-2-ol; 4-(Benzothiazol-6-ylamino)-6-(quinolin-5-ylamino)-[1,3,5]triazin-2-ol; 4-(Benzothiazol-6-ylamino)-6-(4-hydroxy-naphthalen-1-ylamino)-[1,3,5]triazin-2-ol; 4-(Benzothiazol-6-ylamino)-6-(1H-indazol-6-ylamino)-[1,3,5]triazin-2-ol; 4-(Benzothiazol-6-ylamino)-6-[(1H-indazol-6-yl)-methylamino]-[1,3,5]triazin-2-ol; 4-(Benzothiazol-6-ylamino)-6-(1-methyl-1H-indazol-6-ylamino)-[1,3,5]triazin-2-ol; 4-(Benzothiazol-6-ylamino)-6-(6-hydroxy-naphthalen-1-ylamino)-[1,3,5]triazin-2-ol; 4-(Benzothiazol-6-ylamino)-6-(3-hydroxy-phenylamino)-[1,3,5]triazin-2-ol; 4-(Benzothiazol-6-ylamino)-6-[2-(2-hydroxyethyl)-phenylamino]-[1,3,5]triazin-2-ol; 4-(Benzothiazol-6-ylamino)-6-(5-thiophen-2-yl-2H-pyrazol-3-ylamino)-[1,3,5]triazin-2-ol; 4-(Benzothiazol-6-ylamino)-6-(2-phenyl-2H-pyrazol-3-ylamino)-[1,3,5]triazin-2-ol; 4-(Benzothiazol-6-ylamino)-6-(2,4-difluoro-benzylamino)-[1,3,5]triazin-2-ol; 4-(Benzothiazol-6-ylamino)-6-phenylamino-[1,3,5]triazin-2-ol; 4-(1H-Indazol-6-ylamino)-6-(1-methyl-1-phenylethylamino)-[1,3,5]triazin-2-ol; 4-(Benzothiazol-6-ylamino)-6-(2-hydroxy-1-phenylethylamino)-[1,3,5]triazin-2-ol; 4-(1H-Indazol-5-ylamino)-6-(1-methyl-1-phenylethylamino)-[1,3,5]triazin-2-ol; 4-(Benzothiazol-7-ylamino)-6-(1-methyl-1-phenylethylamino)-[1,3,5]triazin-2-ol; 4-(Benzothiazol-6-ylamino)-6-[(furan-2-yl-methyl)amino]-[1,3,5]triazin-2-ol; 4-(Benzothiazol-6-ylamino)-6-[(thiophen-2-yl-methyl)amino]-[1,3,5]triazin-2-ol; 4-(Benzothiazol-6-ylamino)-6-[(furan-3-ylmethyl)-amino-[1,3,5]triazin-2-ol; 4-(Benzothiazol-6-ylamino)-6-[(thiophen-3-yl-methyl)amino]-[1,3,5]triazin-2-ol; 4-(Benzothiazol-6-ylamino)-6-(benzyl-pyrrolidin-3-ylamino)-[1,3,5]triazin-2-ol; 3-{[4-(Benzothiazol-6-ylamino)-6-hydroxy-[1,3,5]triazin-2-yl]-benzylamino}-propane-1,2-diol; 4-(Benzothiazol-6-ylamino)-6-[benzyl-(3-morpholin-4-ylpropyl)-amino]-[1,3,5]triazin-2-ol; 4-(Benzothiazol-6-ylamino)-6-{benzyl-[3-(4-methyl-piperazin-1-yl)-propyl]-amino}-[1,3,5]triazin-2-ol; 4-(Benzothiazol-6-ylamino)-6-[benzyl-(3-dimethylamino-propyl)-amino]-[1,3,5]triazin-2-ol; 4-(Benzothiazol-6-ylamino)-6-[benzyl-(2-piperazin-1-ylethyl)-amino]-[1,3,5]triazin-2-ol; 4-(Benzothiazol-6-ylamino)-6-[benzyl-(2-morpholin-4-ylethyl)-amino]-[1,3,5]triazin-2-ol; 4-(Benzothiazol-6-ylamino)-6-[benzyl-(2-dimethylamino-ethyl)-amino]-[1,3,5]triazin-2-ol; 4-(2-Amino-benzothiazol-6-ylamino)-6-(1-methyl-1-phenylethylamino)-[1,3,5]triazin-2-ol; 4-(1-Methyl-1-phenylethylamino)-6-(quinolin-6-ylamino)-[1,3,5]triazin-2-ol; 4-(Quinolin-6-iylamino)-6-(N-ethylbenzylamino)-[1,3,5]triazin-2-ol; 4-(Quinolin-6-ylamino)-6-(N-methylbenzylamino)-[1,3,5]triazin-2-ol; 4-(Quinolin-6-ylamino)-6-(1-methyl-1-phenylethylamino)-[1,3,5]triazin-2-ol; N-[4-(Benzothiazol-6-ylamino)-6-(1-methyl-1-phenylethylamino)-[1,3,5]triazin-2-yl]-hydroxylamine; 4-(Benzothiazol-6-ylamino)-6-[(4-fluoro-3-trifluoromethylbenzyl)amino]-[1,3,5]triazin-2-ol; 4-(Quinolin-6-ylamino)-6-[(4-fluoro-3-trifluoromethylbenzyl)amino]-[1,3,5]triazin-2-ol; 4-(Benzothiazol-6-ylamino)-6-(ethyl-(pyridin-2-ylmethyl)amino)-[1,3,5]triazin-2-ol; 4-(Benzothiazol-6-ylamino)-6-(N-benzylisopropylamino)-[1,3,5]triazin-2-ol; 4-(Benzothiazol-6-ylamino)-6-(ethyl-(2-fluorobenzyl)amino]-[1,3,5]trizin-2-ol; 4-(Benzothiazol-6-ylamino)-6-[benzyl-(2,2,2-trifluoroethyl)amino]-[1,3,5]triazin-2-ol; 3-[[4-(Benzothiazol-6-ylamino)-6-hydroxy-[1,3,5]triazin-2-yl]-(1-phenylethyl)amino]propane-1,2-diol; 4-(Benzothiazol-6-ylamino)-6-(ethyl-(pyridin-2-ylmethyl)amino)-[1,3,5]triazin-2-ol; 4-(Benzothiazol-6-ylamino)-6-(N-(2-fluorobenzyl)isopropylamino)-[1,3,5]triazin-2-ol; 4-(Benzothiazol-6-ylamino)-6-[ethyl-(1H-indazol-6-yl)amino]-[1,3,5]triazin-2-ol; 4-(Benzothiazol-6-ylamino)-6-{benzyl-[2-(3H-imidazol-4-yl)ethyl]amino}-[1,3,5]triazin-2-ol; 4-(Benzothiazol-6-ylamino)-6-{2-fluorobenzyl-[2-(3H-imidazol-4-yl)ethyl]amino}-[1,3,5]triazin-2-ol; 4-(Benzothiazol-6-ylamino)-6-[benzyl-(3-imidazol-1-yl-propyl)amino]-[1,3,5]triazin-2-ol; 4-{[4-(Benzothiazol-6-ylamino)-6-hydroxy-[1,3,5]triazin-2-yl]-benzylamino}butyric acid; 4-(Benzothiazol-6-ylamino)-6-{(2-piperazin-1-ylethyl)-quinolin-5-ylamino}-[1,3,5]triazin-2-ol; 4-(Benzothiazol-6-ylamino)-6-{benzyl-[2-(3H-imidazol-4-yl)ethyl]amino}-[1,3,5]triazin-2-ol; 4-(Benzothiazol-6-ylamino)-6-(N-benzylpropylamino)-[1,3,5]triazin-2-ol and pharmaceutically acceptable salts thereof.

It is expected that the preferred compounds of Formula II will have similar or identical R₁, R₂, R₃, A₁ and A₂ substituents as compared to the preferred compounds of Formula I.

Preferred compounds of Formula III include 4-(Benzothiazol-6-ylamino)-6-(2-methyl-pyrrolidin-1-yl)-[1,3,5]triazin-2-ol; 4-(Benzothiazol-6-ylamino)-6-(2-benzyl-pyrrolidin-1-yl)-[1,3,5]triazin-2-ol; 4-(Benzothiazol-6-ylamino)-6-(2,6-dimethyl-piperidin-1-yl)-[1,3,5]triazin-2-ol; 4-(Benzothiazol-6-ylamino)-6-(2,5-dimethyl-pyrrolidin-1-yl)-[1,3,5]triazin-2-ol; 4-(Benzothiazol-6-ylamino)-6-(2-phenyl-pyrrolidin-1-yl)-[1,3,5]triazin-2-ol; 4-(Benzothiazol-6-ylamino)-6-(3-phenyl-thiomorpholin-4-yl)-[1,3,5]triazin-2-ol; 4-(Benzothiazol-6-ylamino)-6-(2-phenyl-thiomorpholin-4-yl)-[1,3,5]triazin-2-ol; 4-(Benzothiazol-6-ylamino)-6-(thiomorpholin-4-yl)-[1,3,5]triazin-2-ol; 4-(Benzothiazol-6-ylamino)-6-(3-methyl-piperidin-1-yl)-[1,3,5]triazin-2-ol; 4-(Benzothiazol-6-ylamino)-6-(morpholin-4-yl)-[1,3,5]triazin-2-ol and pharmaceutically acceptable salts thereof.

It is expected that the preferred compounds of Formula IV will have similar or identical R₁, R₂, R₃, A₁ and A₂ substituents as compared to the preferred compounds of Formula III.

A further embodiment of the invention relates to a novel method (Scheme 2, below) of preparing the compounds of Formulae I and III where R is —OH, comprising the steps of:

a) displacing one of three displaceable groups at the 2-, 4- and 6-positions, respectively, of a 1,3,5-triazine ring with 4-methoxybenzyl alcohol to give a 2-(4-methoxybenzyloxy)-[1,3,5]triazine;

b) displacing the second displaceable group with a primary or secondary alkyl or aromatic amine (i) to give a 4-amino-2-(4-methoxybenzyloxy)-[1,3,5]triazine; and

c) displacing the third displaceable group with a primary or secondary alkyl or aromatic amine (ii) under microwave conditions with concomitant loss of the p-methoxybenzyl group to give a 4,6-diamino-(2-hydroxy)-[1,3,5]triazine.

To prepare the compounds of Formulae II and IV, an additional step to steps a)-c) would be required as follows:

d) adding an acylating, sulfonylating or phosphorylating agent to the 4,6-diamino-(2-hydroxy)-[1,3,5]triazine to give a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

Another embodiment of the invention relates to a novel method (Scheme 3, below) of preparing the compounds of Formulae I and III where R is —OH, comprising the steps of:

aa) displacing one of three displaceable groups at the 2-, 4- and 6-positions, respectively, of a 1,3,5-triazine ring with a primary or secondary alkyl or aromatic amine (i) to give a 2-amino-[1,3,5]triazine;

bb) displacing the second displaceable group with a primary or secondary alkyl or aromatic amine (ii) to give a 2,4-diamino-[1,3,5]triazine; and

cc) displacing the third displaceable group with reagent grade TFA to give a 4,6-diamino-(2-hydroxy)-[1,3,5]triazine.

To prepare compounds of Formulae I and III where R is —NHOH, hydroxylamine hydrochloride rather than water was employed to displace the third displaceable group in step cc) of Scheme 3. The —OH attached to the hydroxylamino nitrogen could then optionally be derivatized appropriately as defined by Rₐ.

To prepare compounds of Formulae II and IV, an additional step to steps aa)-cc) would be required as follows:

dd) adding an acylating, sulfonylating or phosphorylating agent to the 4,6-diamino-(2-hydroxy)-[1,3,5]triazine to give a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

A preferred displaceable group in steps a)-c) and aa)-cc) above is chlorine.

Preferred amines (i) and (ii) include 6-aminobenzthiazole and cumyl amine.

Exemplary acylating agents include, but are not limited to, acetic anhydride and butyryl chloride.

Exemplary sulfonylating agents include, but are not limited to, methanesulfonyl chloride and p-toluenesulfonyl chloride.

Exemplary phosphorylating agents include, but are not limited to, phosphoryl chloride.

The invention also relates to methods of inhibiting protein tyrosine kinase activity in a mammal by administration of a therapeutically effective amount of at least one compound of Formulae I, II, III or IV.

The invention is considered to include the enantiomeric, diastereomeric and tautomeric forms of all compounds of Formulae I, II, III and IV as well as their racemic mixtures. In addition, some of the compounds represented by Formulae I, II, III and IV are prodrugs, i.e., derivatives of an acting drug that possess superior delivery capabilities and therapeutic value as compared to the acting drug. Prodrugs are transformed into active drugs by in vivo enzymatic or chemical processes.

I. Definitions The term “alkyl” refers to both linear and branched chain radicals of up to 12 carbon atoms, unless otherwise indicated, and includes, but is not limited, to methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl.

The term “cycloalkyl” refers to a ring composed of from 3 to 8 carbon atoms. Alkyl substituents may optionally be present on the ring. Examples include cyclopropyl, 1,1-dimethyl cyclobutyl, 1,2,3-trimethylcyclopentyl and cyclohexyl.

The term “heterocyclyl” refers to a nonaromatic ring composed of from 3 to 7 carbon atoms and at least one heteroatom selected from N, O or S. Alkyl substituents may optionally be present on the ring. Examples include tetrahydrofuryl, dihydropyranyl, 2,5-dimethypiperidyl, morpholinyl and piperazinyl.

The term “heterocyclylalkyl” refers to a C₁₋₆ alkyl group containing a heterocyclyl substituent. Examples include dihydropyranylethyl and 2-morpholinylpropyl.

The term “hydroxyalkyl” refers to at least one hydroxyl group bonded to any carbon atom along an alkyl chain.

The term “aminoalkyl” refers to at least one primary or secondary amino group bonded to any carbon atom along an alkyl chain.

The term “polyalkoxyalkyl” refers to long-chain alkoxy compounds and includes polyethylene glycols of discreet or monodispersed sizes.

The term “thioalkyl” refers to at least one sulfur group bonded to any carbon atom along an alkyl chain. The sulfur group may be at any oxidation state and includes sulfoxides, sulfones and sulfates.

The term “carboxyalkyl” refers to at least one carboxylate group bonded to any carbon atom along an alkyl chain. The term “carboxylate group” includes carboxylic acids and alkyl, cycloalkyl, aryl or aralkyl carboxylate esters.

The term “heteroaromatic” or “heteroaryl” refers to 5- to 7-membered mono- or 8- to 10-membered bicyclic aromatic ring systems, any ring of which may consist of from one to four heteroatoms selected from N, O or S where the nitrogen and sulfur atoms can exist in any allowed oxidation state. Examples include benzimidazolyl, benzothiazolyl, benzothienyl, benzoxazolyl, furyl, imidazolyl, isothiazolyl, isoxazolyl, oxazolyl, pyrazinyl, pyrazolyl, pyridyl, pyrimidinyl, pyrrolyl, quinolinyl, thiazolyl and thienyl.

The term “heteroaralkyl” refers to a C₁₋₆ alkyl group having a heteroaryl substituent. Examples include furylethyl and 2-quinolinylpropyl.

The term “heteroatom” refers to a nitrogen atom, an oxygen atom or a sulfur atom wherein the nitrogen and sulfur atoms can exist in any allowed oxidation states.

The term “alkoxy” refers to straight or branched chain radicals of up to 12 carbon atoms, unless otherwise indicated, bonded to an oxygen atom. Examples include methoxy, ethoxy, propoxy, isopropoxy and butoxy.

The term “aryl” refers to monocyclic or bicyclic aromatic ring systems containing from 6 to 12 carbons in the ring and optionally substituted with 1-3 substituents selected from alkoxy, alkyl, halogen, hydroxy and heteroaryl. Examples include benzene, biphenyl and napththalene.

The term “aralkyl” refers to a C₁₋₆ alkyl group containing an aryl substituent. Examples include benzyl, phenylethyl or 2-naphthylmethyl.

The term “acyl” refers to the group —C(O)Rₐ, where Rₐ is alkyl, aryl, aralkyl, heteroaryl and heteroaralkyl. An “acylating agent” adds the —C(O)Rₐ group to a molecule.

The term “sulfonyl” refers to the group —S(O)₂Rₐ, where Rₐ is alkyl, aryl, aralkyl, heteroaryl and heteroaralkyl. A “sulfonylating agent” adds the —S(O)₂Rₐ group to a molecule.

The term “phosphoryl” refers to the group —P(O)₂ORₐ, where Rₐ is H, alkyl, aryl, aralkyl, heteroaryl and heteroaralkyl. A “phosphorylating agent” adds the —P(O)₂ORₐ group to a molecule.

II. Therapeutic Uses

The compounds of Formulae I, II, III and IV represent novel potent inhibitors of protein tyrosine kinases and may be useful in the prevention and treatment of disorders resulting from actions of these kinases.

The invention also provides methods of inhibiting a protein tyrosine kinase comprising contacting the protein tyrosine kinase with an effective inhibitory amount of at least one of the compounds of Formulae I, II, III or IV. The protein tyrosine kinases which may be inhibited include, but are not limited to, VEGFR-2 (KDR), c-fins, c-met and tie-2 kinases.

In various embodiments of the invention, the protein tyrosine kinases inhibited by the compounds of Formulae I, II, III or IV are located in cells, in a mammal or in vitro. In the case of mammals, which includes humans, a therapeutically effective amount of a pharmaceutically acceptable form of at least one of the compounds of Formulae I, II, III or IV is administered.

The invention further provides methods of treating cancer in mammals, including humans, by administration of a therapeutically effective amount of a pharmaceutically acceptable composition of least one compound of Formulae I, II, III or IV. Exemplary cancers include, but are not limited to, breast cancer, colon cancer, stomach cancer, hairy cell leukemia and non-small lung carcinoma. In one embodiment of the invention, an effective amount of at least one compound of Formulae I, II, III or IV is administered in combination with an effective amount of a chemotherapeutic agent.

The invention also provides methods of treating vascular diseases, ocular diseases and restenosis in mammals, including humans, by administration of a therapeutically effective amount of a pharmaceutically acceptable form of at least one of the compounds of Formulae I, II, III or IV.

When employed as protein tyrosine kinase inhibitors, the compounds of the invention may be administered in an effective amount within the dosage range of about 0.5 mg to about 10 g, preferably between about 0.5 mg to about 5 g in single or divided daily doses. The dosage administered will be affected by factors such as the route of administration, the health, weight and age of the recipient, the frequency of the treatment and the presence of concurrent and unrelated treatments.

The compounds of Formulae I, II, III and IV may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formulae I, II, III and IV include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamine salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

III. Methods of Preparation

The compounds of Formulae I, II, III and IV may be prepared by either conventional solid phase support methodology or by novel solution-phase synthesis. Scheme 1 is representative of the solid phase support steps utilized to produce compounds of Formulae I and III where R is —OH:

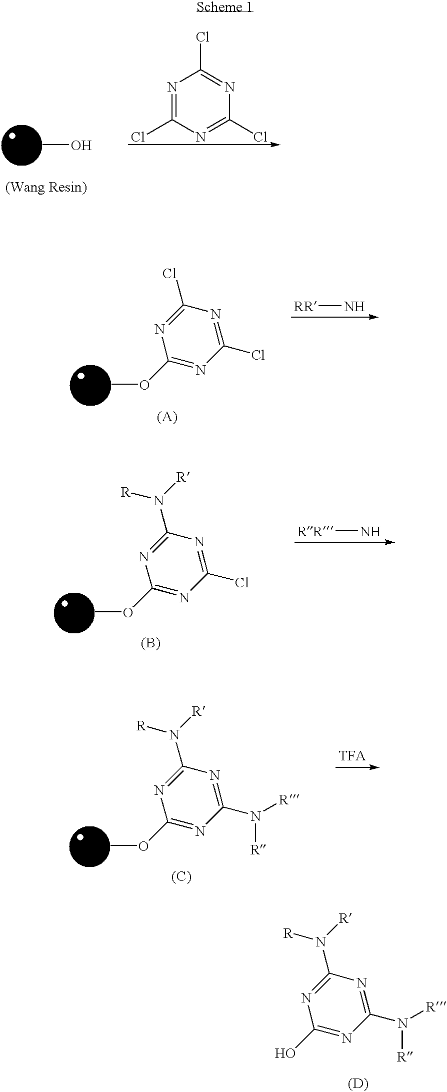

As shown in Scheme 1, Wang resin was treated with excess cyanuric chloride in the presence of base to obtain the resin-bound [1,3,5]triazine ether (A). Resin (A) was then treated with a primary or secondary alkyl or aromatic amine (RR′NH) to yield the resin-bound 4-amino-[1,3,5]triazine ether (B). Resin (B) was then treated with a primary or secondary amine (R″R′″H) to provide the resin-bound 4,6-diamino-[1,3,5]triazine ether (C). Cleavage of the bound 4,6-diamino-[1,3,5]triazine from the resin with trifluoroacetic acid (TFA) yielded the 4,6-diamino-(2-hydroxy)-[1,3,5]triazine (D) in solution as its TFA salt.

To prepare compounds of Formulae II and IV, the 4,6-diamino-(2-hydroxy)-[1,3,5]triazine (D) would be treated with an acylating, sulfonylating or phosphorylating agent to provide a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

Novel solution phase syntheses of the compounds of Formulae I, II, III and IV typically proceed by either one of two routes. In the first route, as represented by Scheme 2, cyanuric chloride is treated with 4-methoxybenzyl alcohol to provide a 4,6-dichloro-2-(4-methoxybenzyloxy)-[1,3,5]triazine which is then treated with primary or secondary alkyl or aromatic amine (RR′NH) followed by another primary or secondary alkyl or aromatic amine (R″R′″NH) to provide, after concomitant loss of the O-4-methoxybenzyl protecting group, a 4,6-diamino-(2-hydroxy)-[1,3,5]triazine of Formulae I and III where R is —OH.

To prepare compounds of Formulae II and IV, the 4,6-diamino-(2-hydroxy)-[1,3,5]triazine would be further treated with an acylating, sulfonylating or phosphorylating agent to provide a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

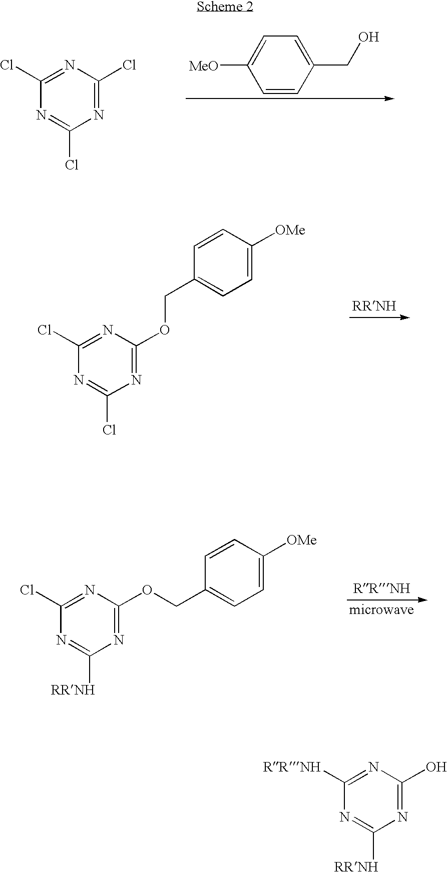

In another novel solution phase route to the compounds of Formulae I, II, III and IV as represented by Scheme 3, cyanuric chloride is treated sequentially with primary or secondary alkyl or aromatic amines (RR′NH and R″R′″ NH), to provide a 6-chloro-2,4-diamino-[1,3,5]triazine which is treated with reagent grade trifluoroacetic acid to provide, after neutralization, a 4,6-diamino-(2-hydroxy)-[1,3,5]triazine compound of Formulae I and III where R is —OH.

To prepare the compounds of Formulae I and III where R is —NHORₐ, the 6-chloro-2,4-diamino-[1,3,5]triazine was treated with hydroxylamine rather than TFA in the final step of Scheme III.

To prepare compounds of Formulae II and IV, the 4,6-diamino-(2-hydroxy)-[1,3,5]triazine (D) would be further treated with an acylating, sulfonylating or phosphorylating agent to provide a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

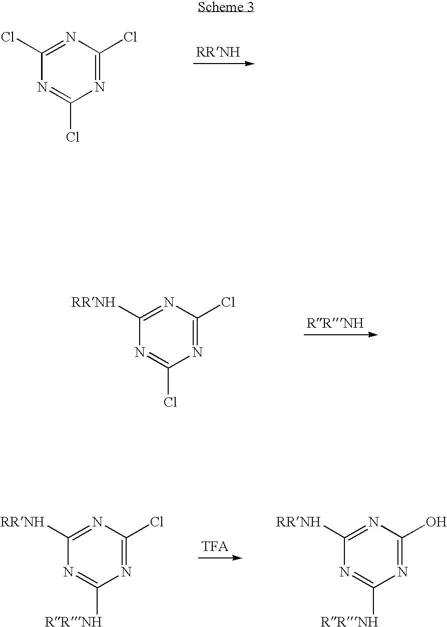

The Scheme 2 approach to preparing the protein kinase inhibitors of Formulae I, II, m and IV is ideal for acid-sensitive compounds because each step in the synthesis occurs under basic conditions. The final step of the Scheme 3 route is superior to similar conversions reported in the literature in that it is carried out under very mild reaction conditions of room temperature and reaction times of less than 15 minutes.

### EXPERIMENTAL

The following examples below are intended to illustrate, not limit, the invention.

The following reagents were used in the examples:

Wang Resin, a solid support, is sold by Polymer Labs, Amherst, Mass.

Cyanuric chloride, a trichloro[1,3,5]triazine, is sold by Aldrich Chemical Company, Milwaukee, Wis.

Diisopropylethylamine (DIEA), trifluoroacetic acid (TFA) and all anhydrous solvents, such as tetrahydrofuran (THF) and dichloromethane (DCM), were purchased from Aldrich Chemical Co (Milwaukee, Wis.) and were used as such without further purification.

Amines for use as substituents on the triazine ring were purchased from various chemical suppliers including Aldrich, Lancaster, TCI, Maybridge and Acros and were used as such without additional purification or were synthesized according to standard literature procedures.

I. General Procedure for Solid Phase Supported Synthesis (Scheme 1):

A solution of cyanuric chloride (22.8 g, 122 mmol) in 180 mL of anhydrous tetrahydrofuran (THF) was added in one portion to Wang resin (12 g, 20 mmol) under a nitrogen atmosphere. The resulting suspension was shaken at room temperature (rt) for 15 min. Diisopropylethylamine (DIEA) (21.6 mL, 122 mmol) was added slowly via syringe to the mixture followed by shaking of the suspension at rt for 18 h. The suspension was filtered and the resin washed sequentially with THF and dichloromethane (DCM). The resin was then dried in vacuo to yield 15 g of a pale yellowish resin (A) (100% yield based on weight and original loading of 1.7 mmol/g).

To the resin (A) was added a solution of a primary or secondary aromatic or alkylamine (100 mmol) in 150 mL of anhydrous THF under a nitrogen atmosphere. The resulting suspension was shaken at rt for 18 h, filtered, and the resin washed sequentially with THF, DCM, methanol (MeOH) and DCM. The resin was then dried in vacuo to yield a deep yellowish resin (B) (100% yield based on weight and original loading of 1.7 mmol/g).

The resin (B) (0.17 mmol) was equally apportioned into several vials. To each vial was added 2 mL of a different amine solution (0.25 M) in dioxane and 100 μl of DIEA and the vials were sealed and the resins heated and stirred at 90 degrees for 16 h. After being allowed to cool to room temperature, the vials were opened and the resin in each vial was separately filtered and washed sequentially with MeOH and DCM. Each resin (C) was then dried in vacuo.

To each portion of resin (C) above in a vial was added 2 mL of 5-50% trifluoroacetic acid (TFA)/DCM. The vials were sealed and allowed to stand at rt with occasional manual shaking for 3 h. The vials were opened and the resin in each vial was separately filtered and washed with a 0.5 mL portion of TFA/DCM. The filtrates and washings were collected for each vial and were concentrated in vacuo for 12 h. Each of the resulting compounds (D) was analyzed by LC/MS and ¹H-NMR.

### EXAMPLE 1

4-(Benzothiazol-6-ylamino)-6-(N-ethylbenzylamino)-[1,3,5]triazin-2-ol

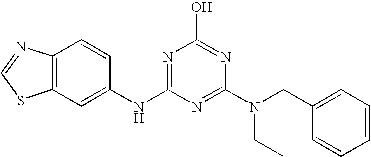

The procedure followed was that described for Scheme 1. Yield was 250 mg (82%). MS: 379 (M+1). LC/MS purity: 97%. ¹H-NMR (300 MHz, DMSO-d₆): δ 9.4 (d, 1H); 8.2 (s, 1H); 8.0 (d, 1H); 7.5 (d, 1H); 7.4 (m, 5H); 4.8 (s, 2H); 3.6 (m, 2H); 1.1 (t, 3H).

### EXAMPLE 2

4-(Benzothiazol-6-ylamino)-6-(benzylamino)-[1,3,5]triazin-2-ol

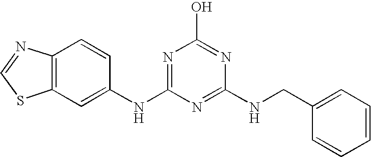

The procedure followed was that described for Scheme I. Yield was 104 mg (88%). MS: 351 (M+1). LC/MS purity: 98%.

### EXAMPLE 3

(R)-4-(Benzothiazol-6-ylamino)-6-(1-phenylethylamino)-[1,3,5]triazin-2-ol

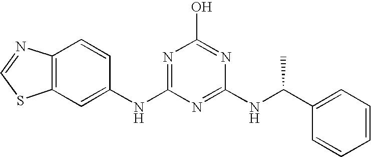

The procedure followed was that described for Scheme I. Yield was 116 mg (96%). MS: 365 (M+1). LC/MS purity: 98%.

### EXAMPLE 4

(S)-4-(Benzothiazol-6-ylamino)-6-(1-phenylethylamino)-[1,3,5]triazin-2-ol

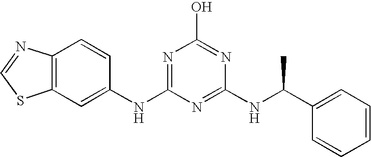

The procedure followed was that described for Scheme I. Yield was 114 mg (95%). MS: 365 (M+1). LC/MS purity: 99%.

### EXAMPLE 5

4-(Benzothiazol-6-ylamino)-6-(1-methyl-1-phenylethylamino)-1,3,5]triazin-2-ol

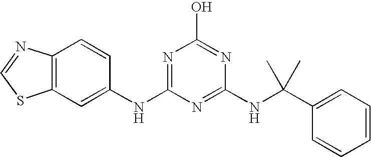

The procedure followed was that described for Scheme I. Yield was 104 mg (85%). MS: 379 (M+1). LC/MS purity: 97%.

### EXAMPLE 6

4-(Benzothiazol-6-ylamino)-6-[methyl-(2-pyridyl-2-ylethyl)amino]-[1,3,5]triazin-2-ol

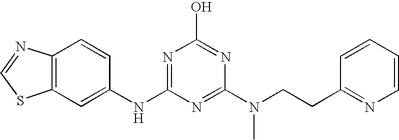

The procedure followed was that described for Scheme I. Yield was 9.91 mg (>80%). MS: 380 (M+1). LC/MS purity: 94%.)

### EXAMPLE 7

4-(1-methyl-1-phenylethylamino)-6-(guinolin-6-ylamino)-[1,3,5]triazin-2-ol

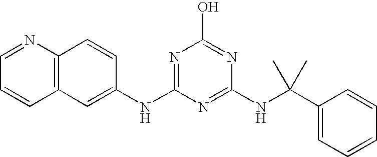

The procedure followed was that described for Scheme I. Yield was 6.4 mg (>80%). MS: 373 (M+1). LC/MS purity: 87%.

### EXAMPLE 8

4-(Benzothiazol-6-ylamino)-6-(2-phenyl-pyrrolidin-1-yl)-[1,3,5]triazin-2-ol

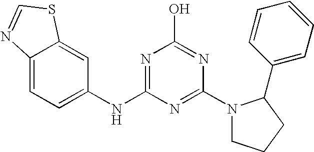

The procedure followed was that described for Scheme I. Yield was 13 mg (80%). MS: 391 (M+1). LC/MS purity: 88%.

### EXAMPLE 9

4-(Benzothiazol-6-ylamino)-6-(2-phenyl-thiomorpholin-4-yl)-[1,3,5]triazin-2-ol

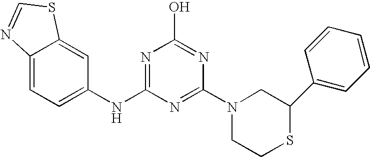

The procedure followed was that described for Scheme I. Yield was 9.1 mg (>80%). MS: 423 (M+1). LC/MS purity: 97%.

### EXAMPLE 10

4-(Benzothiazol-6-ylamino)-6-(3-phenyl-thiomorpholin-4-yl)-[1,3,5]triazin-2-ol

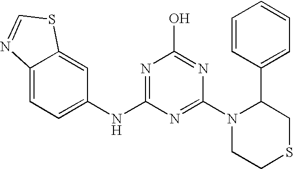

The procedure followed was that described for Scheme I. Yield was 14 mg (>80%). MS: 423 (M+1). LC/MS purity: 87%.

### EXAMPLE 11

3-(Benzothiazol-6-ylamino)-5-[(1H-indazol-6-yl)-methylamino]-phenol

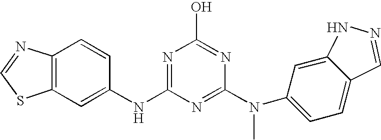

The procedure followed was that described for Scheme I. Yield was 34 mg (79%). MS: 391 (M+1). LC/MS purity: 99%.

II. Exemplary Procedure for Solution Phase Synthesis (Scheme 2):

### EXAMPLE 12

4-(Benzothiazol-6-ylamino)-6-(1-methyl-1-phenylethylamino)-[1,3,5]triazin-2-ol

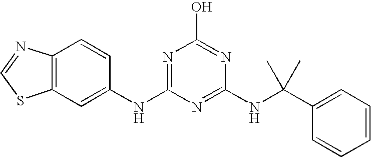

A solution of cyanuric chloride (456 mg, 2.48 mmol), 4-methoxybenzyl alcohol (557 mg, 4.03 mmol), and DIEA (371 mg, 2.9 mmol) in THF (8 mL) was stirred for 15 min. The resultant cloudy suspension was diluted with H₂O (100 mL) and the reaction was extracted with DCM (2×20 mL). The combined organic layers were dried over MgSO₄, filtered and concentrated. Chromatography on silica gel (hexanes:EtOAc, 1:1) yielded 2,4-dichloro-6-(4-methoxybenzyloxy)-[1,3,5]triazine (256 mg, 1.25 mmol). A solution of 2,4-dichloro-6-(4-methoxybenzyloxy)-[1,3,5]triazine (613 mg, 2.15 mmol), cumyl amine (285 mg, 2.11 mmol) and DIEA (267 mg, 2.07 mmol) in THF (13 mL) was stirred at rt for 5 minutes. The resultant cloudy suspension was diluted with H₂O (150 mL), and extracted with DCM (2×30 mL). The combined organic layers were dried over MgSO₄, filtered and concentrated. Chromatography on silica gel (hexanes:EtOAc, 85:15) yielded [4-chloro-6-(4-methoxybenzyloxy)-[1,3,5]triazin-2-yl]-(1-methyl-1-phenylethyl)amine (512 mg, 1.33 mmol). A solution of [4-chloro-6-(4-methoxybenzyloxy)-[1,3,5]triazin-2-yl]-(1-methyl-1-phenylethyl)amine (50 mg, 0.13 mmol), 6-aminobenzothiazole (25 mg, 0.16 mmol) and DIEA (18 mg, 0.14 mmol) in THF (2 mL) was prepared in a sealed tube and heated to 120° C. in a microwave reactor (Personal Chemistry, Smith Synthesizer) for 90 min. Chromatography on silica gel (DCM:MeOH, gradient 19:1, 9:1) yielded 4-(benzothiazol-6-yl-amino)-6-(1-methyl-1-phenylethylamino)-[1,3,5]triazin-2-ol (3 mg, 8×10-3 mmol). MS: 379 (M+1). LC/MS purity: 97%.

III. Exemplary Procedure for Solution Phase Synthesis (Scheme 3):

### EXAMPLE 13

4-(Benzothiazol-6-ylamino)-6-(1-methyl-1-phenylethylamino)-[1,3,5]triazin-2-ol

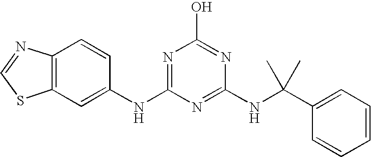

A solution of cyanuric chloride (737 mg, 4.0 mmol) in THF (10 mL) was treated with 6-aminobenzothiazole (533 mg, 3.5 mmol) and DIEA (0.70 mL, 4.0 mmol). After 30 min of stirring, the resulting suspension was poured into 800 mL H₂O and 100 mL DCM. The reaction was partitioned and the organic layer was dried over MgSO₄, filtered, and concentrated. Without further purification, the resulting benzothiazol-6-yl-(4,6-dichloro-[1,3,5]triazin-2-yl)-amine was dissolved in THF (12 mL), and treated with cumylamine (450 mg, 3.3 mmol) followed by DIEA (0.7 mL, 4.0 mmol). After 10 min of stirring, the reaction was poured into 250 mL H₂O and 20 mL DCM. The reaction was partitioned and the aqueous layer was extracted with an additional 20 mL DCM. The combined organic layers were dried over MgSO₄, filtered and concentrated. Chromatography on silica gel (hexanes: EtOAc, 2:1) gave 308 mg of N-(benzothiazol-6-yl)-6-chloro-N′-(1-methyl-1-phenylethyl)-[1,3,5]triazine-2,4-diamine. MS: 397 (M+1). LC/MS purity: 100%.

A solution of N-(benzothiazol-6-yl)-6-chloro-N-(1-methyl-1-phenylethyl)-[1,3,5]triazine-2,4-diamine (5.4 mmol) was dissolved in 50 mL DCM and 5 mL TFA and treated with 5 mL H₂O. The reaction was allow to stir at rt for 48 h to provide 4-(benzothiazol-6-yl-amino)-6-(1-methyl-1-phenylethylamino)-[1,3,5]triazin-2-ol as the tri-TFA salt. MS: 379+3 TFA (M+1). LC/MS purity: 100%.

### EXAMPLE 14

N-[4-Benzothiazol-6-ylamino)-6-(1-methyl-1-phenylethylamino)-[1,3,5]triazin-2-yl]-hydroxylamine

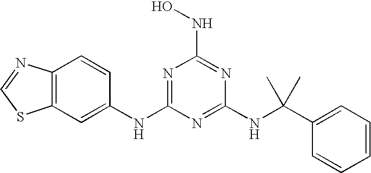

The procedure followed was that described for Scheme III except for the final step. The displacement of the chlorine on the triazine ring with reagent TFA was replaced by the following step:

A solution of hydroxylamine hydrochloride (69 mg, 1.0 mmol) in ethanol (10 mL) and DIEA (1 mL) was treated with N-(benzothiazol-6-yl)-6-(chloro-N′-(1-methyl-1-phenylethyl)-[1,3,5]triazine-2,4-diamine (95 mg, 0.24 mmol). The solution was heated to 75° C. for 16 hrs. After cooling to room temperature, the solution was evaporated onto celite (10 g) and chromatographed on silica gel (95/5 DCM/MeOH) to give 15 mg (0.04 mmol) of N-[4-(benzothiazol-6-yl-amino)-6-(1-methyl-1-phenylethylamino)-[1,3,5]triazin-2-yl]-hydroxylamine. MS 393 (M+i). LC/MS purity: 100%. ¹H-NMR (300 MHz, acetone-d₆): δ 9.0 (s, 1H); 7.2-8.0 (m, 8H); 1.8 (s, 6H).

IV. Comparative Examples

### EXAMPLE 15

N-(Benzothiazol-6-yl)-N′-(1-methyl-1-phenylethyl)-[1,3,5]triazine-2,4,6-triamine

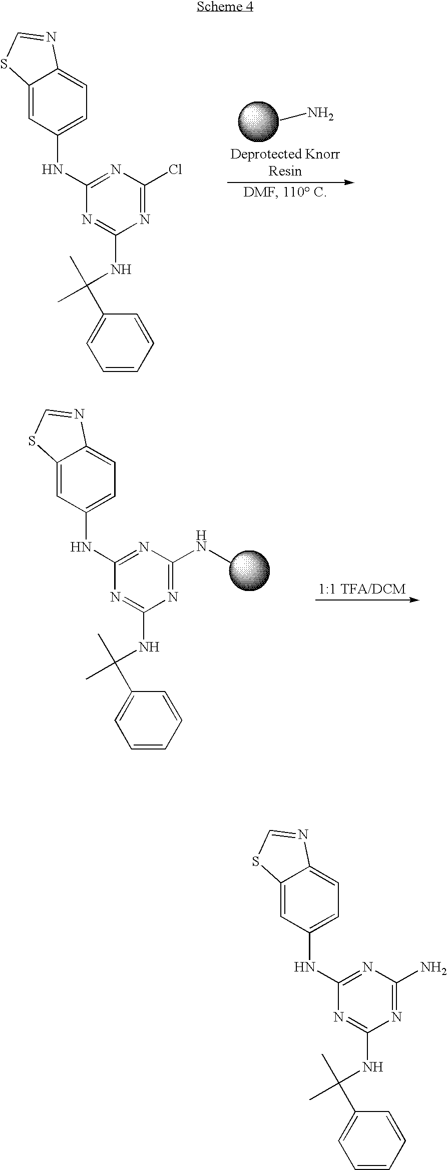

Knorr resin (350 mg, 0.25 mmol) was treated with a solution of 20% piperidine in DMF and shaken for 120 minutes. The resin was rinsed three times alternately with DCM (10 mL) and MeOH (10 mL). The resin was re-swollen with DCM (1.5 mL) and treated with a solution of N-benzothiazol-6-yl-6-chloro-N′-(1-methyl-1-phenylethyl)-[1,3,5]triazine-2,4-diamine (100 mg, 0.25 mmol) in DMF (2.5 mL). The stirred suspension was heated at 110° C. in a sealed vial for 16 hrs. Cleavage from the resin was effected with 50/50 (v/v) TFA/DCM. The resulting solution was concentrated, dissolved in MeOH, and chromatographed on a TLC prep plate using (DCM:MeOH, 9:1) to give 1.5 mg N-(benzothiazol-6-yl)-N′-(1-methyl-1-phenylethyl)-[1,3,5]triazine-2,4,6-triamine as its TFA salt. MS: 378 (M+1). LC/MS purity: 87%.

### EXAMPLE 16

N-(Benzothiazol-6-yl)-N′-(1-methyl-1-phenylethyl)-[1,3,5]triazine-2,4-diamine

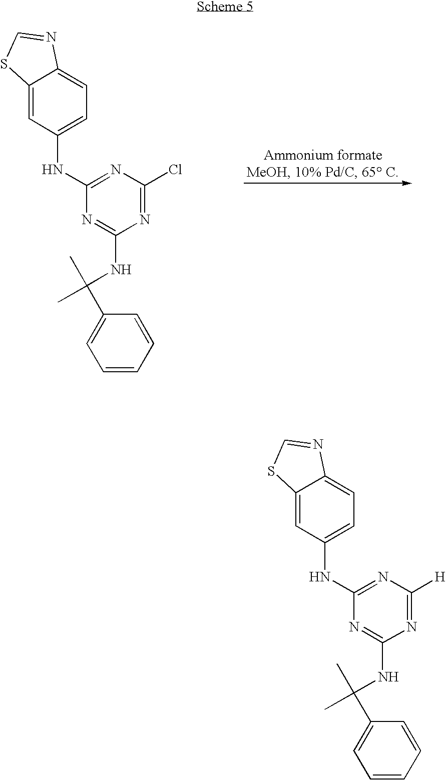

A solution of N-(benzothiazol-6-yl)-6-chloro-N′-(1-methyl-1-phenylethyl)-[1,3,5]triazine-2,4-diamine (100 mg, 0.25 mmol) in MeOH (3 mL) was treated with ammonium formate (350 mg, 5.2 mmol) and 5% Pd/C (43 mg, 0.25 mmol) and heated to 65° C. for 16 hrs. Chromatography of the resulting mixture (DCM:MeOH, 9:1) gave the free base of N-(benzothiazol-6-yl)-N′-(1-methyl-1-phenylethyl)-[1,3,5]triazine-2,4-diamine. The material was dissolved in 50/50 (v/v) TFA/DCM (1 mL) and concentrated to give 2.9 mg of the TFA salt. MS: 363 (M+1). LC/MS purity: 100%.

### EXAMPLE 17

N-(Benzothiazol-6-yl)-6-methoxy-N′-(1-methyl-1-1-phenylethyl)-[1,3,5]triazine-2,4-diamine

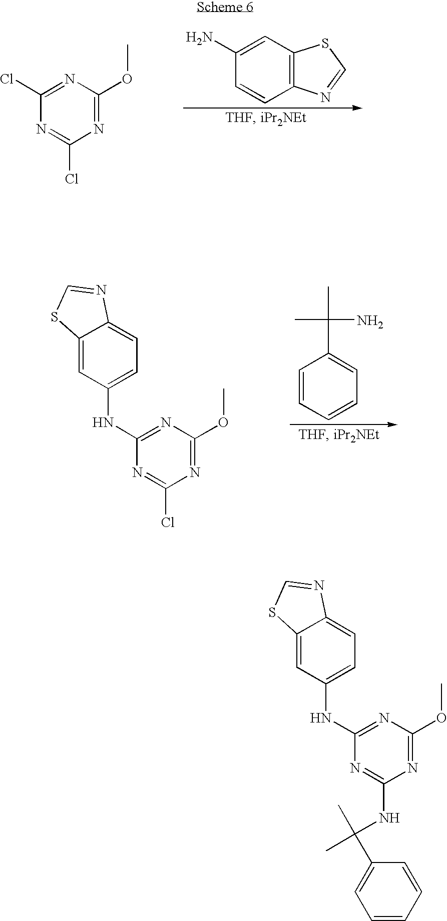

A solution of 2,4-dichloro-6-methoxy-[1,3,5]triazine (328 mg, 2 mmol), and 6-aminobenzothiazole (300 mg, 2 mmol) in THF (10 mL) was treated with DIEA (0.36 mL, 2 mmol) and stirred for 1 hr. The resulting suspension was poured into H₂O (50 mL) and DCM (50 mL) and partitioned. The aqueous layer was washed with 50 mL DCM and the combined organic layers were dried over MgSO₄, filtered and concentrated. Chromatography on silica gel (Hexanes: EtOAc 2:1) gave N-(benzothiazol-6-yl)-(4-chloro-6-methoxy-[1,3,5]triazin-2-yl)-amine (130 mg, 0.44 mmol). A solution of 1-(6-amino-benzothiazolyl)-3-chloro-5-methoxytriazine (130 mg, 0.44 mmol) in dioxane (10 mL) was treated with cumylamine (65 mg, 0.48 mmol) and DIEA (89 mg, 0.69 mmol) and heated to reflux for 16 hrs. The reaction was cooled to rt, poured into H₂O (50 mL) and extracted with DCM (2×50 mL). The combined organic layers were dried over MgSO₄, filtered and concentrated. Chromatography on silica gel (hexanes: EtOAc, 1:1) gave 110 mg N-(benzothiazol-6-yl)-6-methoxy-N′-(1-methyl-1-phenylethyl)-[1,3,5]triazine-2,4-diamine. MS: 393 (M+1). LC/MS purity: 99%.

### EXAMPLE 18

N-(Benzothiazol-6-yl)-6-benzyloxy-N′-(1-methyl-1-phenylethyl)-[1,3,5]triazine-2,4-diamine

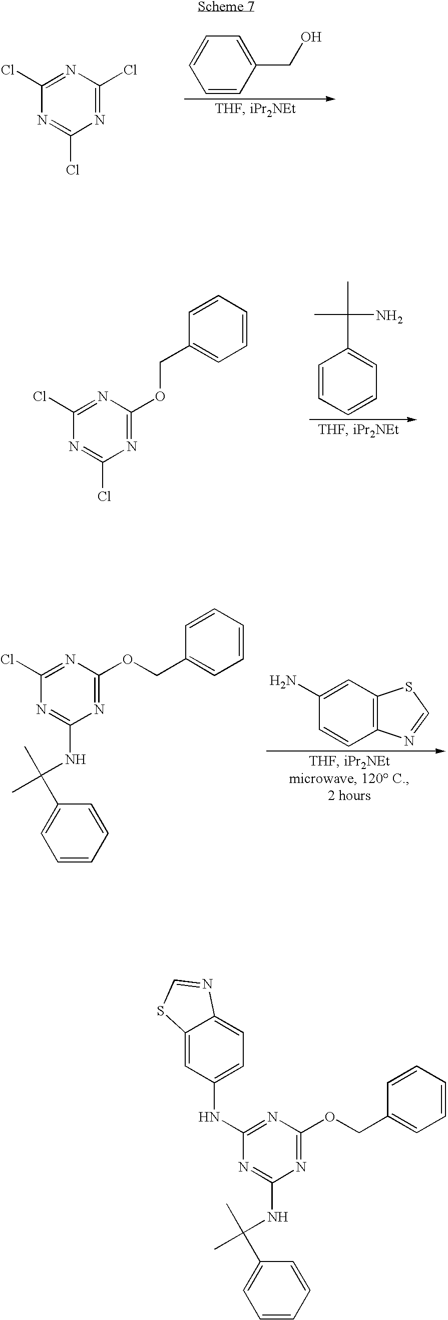

A solution of cyanuric chloride (1.0 g, 5.4 mmol) in THF (50 mL) at −30° C. was treated with benzyl alcohol (550 mg, 5.12 mmol) and DIEA (1.0 mL, 5.8 mmol). The reaction was allowed to warm to rt over 4 hrs. The yellow reaction mixture was poured onto 150 mL H₂O and 20 mL DCM. The reaction was partitioned and the organic layer was dried over MgSO₄, filtered, and concentrated onto diatomaceous earth. Chromatography on silica gel (hexanes: EtOAc, 9:1) gave 2-benzyloxy-4,6-dichloro-[1,3,5]triazine (620 mg, 2.42 mmol). A solution of 2-benzyloxy-4,6-dichloro-[1,3,5]triazine (256 mg, 1.0 mmol) in THF (10 mL) was treated with cumylamine (138 mg, 1.02 mmol) followed by DIEA (133 mg, 1.04 mmol). The reaction was allowed to stir at rt for 3 hours, and was diluted with H₂O (120 mL), and extracted with DCM (3×30 mL). The combined organic layers were dried over MgSO₄, filtered and concentrated. Chromatography on silica gel (hexanes: EtOAc, 9:1) gave (4-benzyloxy-6-chloro-[1,3,5]triazin-2-yl)-(1-methyl-1-phenylethyl)-amine (83 mg, 0.23 mmol). A solution of (4-benzyloxy-6-chloro-[1,3,5]triazin-2-yl)-(1-methyl-1-phenylethyl)-amine (80 mg, 0.22 mmol), 6-aminobenzothiazole (50 mg, 0.33 mmol), and DIEA (33 mg, 0.26 mmol) in THF (1.75 mL) was heated to 120° C. in the microwave for 12 hrs. The reaction was concentrated, and chromatography on silica gel (hexanes: EtOAc, 1:1) gave N-(benzothiazol-6-yl)-6-benzyloxy-N′-(1-methyl-1-phenylethyl)-[1,3,5]triazine-2,4-diamine (60 mg, 0.13 mmol). MS: 469 (M+1). LC/MS purity: 100%.

V. Results

Two in vitro assays were run to measure potency of protein tyrosine kinase inhibition by selected compounds of Formulae I and III. Compounds of Formulae II and IV are expected to behave like typical prodrugs in that potency measurements will be best reflected by in vivo studies.

KDR Enzymatic Assay. A fluorescence polarization competition immunoassay was used to determine the compound potency for KDR. The assay was performed in black 96-well microplates (LJL BioSystems). The assay buffer used was 100 mM HEPES, pH 7.5, 1 mM DTT, 0.01% (v/v) Tween-20. Compounds were diluted in assay buffer containing 4% DMSO just prior to the assay. To each well, 5 μl of compound were added followed by the addition of 3 μl of a mix containing 33.3 μM ATP (Sigma), 33.3 μg/ml poly(E₄,Y) (Sigma), and 16.7 mM MgCl₂ in assay buffer. The kinase reaction was initiated by adding 2 μl of 8 nM KDR in assay buffer. The final concentrations in the assay were 1.6 nM KDR, 10 μM ATP, 10 μg/ml poly(E₄,Y), 5 mM MgCl₂, 2% DMSO. Control reactions were ran in each plate: in positive and negative control wells assay buffer (made 4% in DMSO) was substituted for the compound; in addition, positive control wells did not receive KDR.

The plates were incubated at room temperature for 5 min. At the end of the incubation, the reaction was quenched with 1.2 μl of 50 mM EDTA. Following a 5-min incubation, each well received 10 μl of a 1:1:3 mixture of anti-phosphotyrosine antibody, 10×, PTK green tracer, 10× (vortexed), FP dilution buffer, respectively (all from PanVera, cat. # P2837). The plate was covered, incubated for 30 min at room temperature and the fluorescence polarization was read on an ANALYST™ HT Assay Detection System (LJL Biosystems, Sunnyvale, Calif.). The instrument settings were: 485 nm excitation filter; 530 nm emission filter; Z height: middle of well; G factor: 0.93. Under these conditions, the fluorescence polarization values for positive and negative controls were ˜260 and ˜110, respectively, and were used to define the 100% and 0% inhibition of the KDR reaction. The IC₅₀ values reported are the averages of three independent measurements.

KDR Cell-Based Assay. To determine the effect of the test compounds on KDR function in cells, VEGF-stimulated MAP kinase activation in human umbilical vein endothelial cells (HUVEC), which express endogenous Flk-1(KDR), was examined. HUVECs were grown to confluence in EMB-2 endothelial cell media (Biowhittaker Inc., Walkersville, Md.) at 37° C. and 5% CO₂. Confluent, quiescent HUVECs were treated with the test compounds 30 minutes prior to stimulation with 25 ng/ml VEGF for 10 minutes at 37° C. These cells were then lysed in HNTG buffer (50 mM HEPES, 150 mM NaCl, 1% triton-X-100, 1.5 mM MgCl₂, 10% glycerol, 10 mM NaF, 1 mM EDTA, 10 mM sodium pyrophosphate, 1 uM PMSF and 250 uM NaVO₄). Cell lysates (40 ug total protein) were separated by SDS-PAGE and transferred to nitrocellulose. Immunoblots were probed with a polyclonal antibody to phosphorylated MAP kinase (Cell Signaling Technologies, Woburn, Mass.) and alkaline phosphatase conjugated secondary antibody (Biorad Labs, Hercules, Calif.). Immunoblot detection was done by measuring the fluorescent product of the alkaline phosphatase reaction with the substrate 9H-(1,3-dichloro-9,9-dimethylacridin-2-one-7-yl) phosphate, diammonium salt (DDAO phosphate) (Molecular Probes, Eugene, Oreg.) using a Molecular Dynamics Typhoon Imaging system (Molecular Dynamics, Sunnyvale, Calif.). Quantitation of DDAO phosphate signal and IC50 determinations were done with Molecular Dynamics ImageQuant software.

As shown in Table 1,4-(Benzothiazol-6-ylamino)-6-(1-methyl-1-phenylethylamino)-[1,3,5]triazin-2-ol (Examples 5, 12 and 13) was one of the most potent protein tyrosine kinase inhibitors tested. Analogous Example 2 with no branching at R₃ was less active than Examples 3 and 4 which each had mono-methyl substitution at R₃. The R-enantiomer (Example 3) was more potent than the corresponding S-enantiomer (Example 4). Examples 9 and 10 are examples of potent inhibitors represented by Formula III. Example 14 is an example of a potent inhibitor that is a hydroxylamine of Formula I. Comparative examples 15-18, where the hydroxy group of one of the most potent compounds (represented by Examples 5, 12 and 13) was replaced by —NH₂, —H, —OCH₃ and —OCH₂Ph, respectively, exhibited decreased inhibition.

TABLE 1

| | | IC50 Cell-Based KDR |
|---|---|---|
| Example | IC50 Enzyme (μM) | Assay (μM) |
| 1 | A | A |
| 2 | B | N.A. |
| 3 | A | B |
| 4 | B | N.A. |
| 5, 12, 13 | A | A |
| 6 | A | B |
| 7 | A | B |
| 8 | A | B |
| 9 | A | B |
| 10 | A | B |
| 11 | A | B |
| 14 | A | B |
| 15 | B | N.A. |
| 16 | B | N.A. |
| 17 | B | N.A. |
| 18 | B | N.A. |

A: <1 μM
B: >1 μM and <15 μM
C: >15 μM and <50 μM
D: >50 μM
N.A. = not available

The claimed invention is:

1. A compound of Formula I: 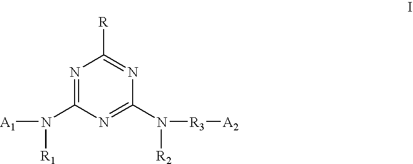
or a solvate, hydrate, tautomer or pharmaceutically acceptable salt thereof, wherein
R is
—OH or —NHORₐ, wherein Rₐ is hydrogen, alkyl, cycloalkyl, aryl or aralkyl;
A₁ is
a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORₐ, —COORₐ, —CONRₐR_{b}, —NHCORₐR_{b}, —NHSO₂Rₐ, —SO₂Rₐ, —SO₃Rₐ or —SO₂NRₐR_{b}, wherein Rₐ and R_{b} are independently hydrogen, alkyl, cycloalkyl, aryl or aralkyl;
R₁ is
hydrogen, alkyl, hydroxy or alkoxy;
R₂ is
hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;
R₃ is
a direct link or
C₁₋₆ alkyl, C₁₋₆ alkoxy, C₁-6 thioalkyl, C₁₋₆ hydroxyalkyl or C₁₋₆ carboxyalkyl; and
A₂ is
phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{c}, —COOR_{c}, —CONR_{c}R_{d}, —N(R₁)COR_{c}, —SO₂R_{c}, —SO₃R_{c} or —SO₂NR_{c}R_{d};
a 5- to 7-membered mono- or a 8- to 10-membered bicyclic heteroaromatic ring having from one to four heteroatoms selected from N, O or S, and may be optionally substituted with C₁₋₆alkyl, amino, halogen, hydroxy, alkoxy, aryloxy, arylalkoxy, —CF₃, —OCF₃, —COR_{c}, —COOR_{c}R_{d}, —CONR_{c}R_{d}, —NHCOR_{c}R_{d}, NHSO₂R_{c}, —SO₂R_{c}, —SO₃R_{c} or —SO₂NR_{c} R_{d}; or
—COR_{c}, —COOR_{c} or —CONR_{c}R_{d}, wherein
R_{c} and R_{d} are independently hydrogen, alkyl, cycloalkyl, aryl, aralkyl, heteroaralkyl or heteroaryl.

2. A compound of Formula II: 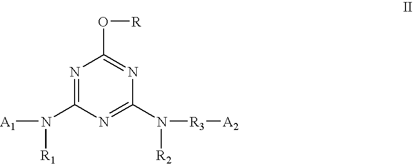
or a solvate, hydrate, tautomer or pharmaceutically acceptable salt thereof, wherein
R is
—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;
A₁ is
a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c}, or —SO₂NR_{c}R_{d}, wherein R_{c} and R_{d} are independently hydrogen, alkyl, cycloalkyl, aryl or aralkyl;
R₁ is
hydrogen, alkyl, hydroxy or alkoxy;
R₂ is
hydrogen, alkyl, carboxyalkyl, cycloalkyl, heterocyclylalkyl, aryl, aralkyl, heteroaryl, heteroaralkyl, hydroxyalkyl, aminoalkyl, hydroxy, alkoxy or polyalkoxyalkyl;
R₃ is
a direct link or
C₁₋₆ alkyl, C₁₋₆ alkoxy, C₁₋₆ thioalkyl, C₁₋₆ hydroxyalkyl or C₁₋₆ carboxyalkyl; and
A₂ is
phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂R_{c}, —SO₃Rₑ or —SO₂NRₑ R_{f};
a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COORₑ, —CONRₑR_{f}, —NHCORₑR_{f}, NHSO₂Rₐ, —SO₂Rₐ, —SO₃Rₐ or —SO₂NRₐR_{b}; or
—CORₑ, —COORₑ or —CONRₑR_{f}, wherein
Rₑ and R_{f} are independently hydrogen, alkyl, cycloalkyl, aryl, aralkyl, heteroaralkyl or heteroaryl.

3. A compound of Formula III: 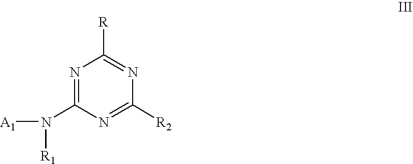
or a solvate, hydrate, tautomer or pharmaceutically acceptable salt thereof, wherein
R is
—OH or —NHORₐ, wherein Rₐ is hydrogen, alkyl, cycloalkyl, aryl or aralkyl;
A₁ is
a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —CORA, —COORₐ, —CONRₐR_{b}, —NHCORₐR_{b}, —NHSO₂Rₐ, —SO₂Rₐ, —SO₃Rₐ or —SO₂NRₐR_{b}, wherein Rₐ and R_{b} are independently hydrogen, alkyl, cycloalkyl, aryl or aralkyl;
R₁ is
hydrogen, alkyl, hydroxy or alkoxy; and 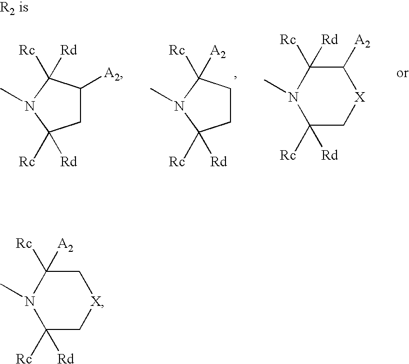
wherein
R_{c} and R_{d} are independently hydrogen or alkyl;
X is N, O or S; and
A₂ is
phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —CORₑ, —COORₑ, —CONRₑR_{f}, —N(R₁)CORₑ, —SO₂Rₑ, —SO₃Rₑ or —SO₂NRₑR_{f}; or
a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —CORₑ, —COO, —CONRₑR_{f}, —NHCORₑR_{f}, NHSO₂Rₑ, —SO₂Rₑ, —SO₃Rₑ or —SO₂NRₑR_{f}, wherein
Rₑ and R_{f} are independently hydrogen, alkyl, cycloalkyl, aryl, aralkyl, heteroaralkyl or heteroaryl.

4. A compound of Formula IV: 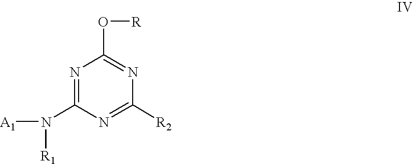
or a solvate, hydrate, tautomer or pharmaceutically acceptable salt thereof, wherein
R is
—CORₐ, —CONRₐR_{b}, —SO₂Rₐ or —PO₃RₐR_{b}, wherein Rₐ and R_{b} are independently hydrogen, alkyl, cycloalkyl, polyalkoxyalkyl, aryl or aralkyl;
A₁ is
a 5- to 6-membered mono- or a 8- to 10-membered bicyclic heteroaromatic ring having from one to four heteroatoms selected from N, O or S, and may be optionally substituted with C₁₋₆ alkyl, amino, alkylamino, halogen, hydroxy, alkoxy, —OCO-alkyl, —OCO-alkylamino, —OCO-alkylamido, aryloxy, arylalkoxy, —CF₃, —OCF₃, —COR_{c}, —COOR_{c}, —CONR_{c}R_{d}, —NHCOR_{c}R_{d}, —NHSO₂R_{c}, —SO₂R_{c}, —SO₃R_{c} or —SO₂NR_{c}R_{d}, wherein R_{c} and R_{d} are independently hydrogen, alkyl, cycloalkyl, aryl or aralkyl;
R₁ is
hydrogen, alkyl, hydroxy or alkoxy; and 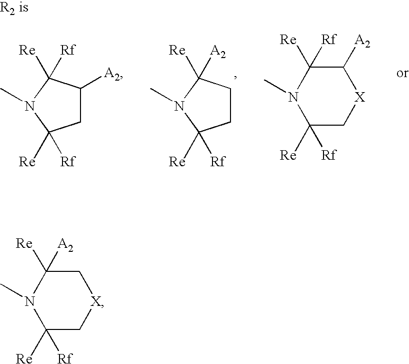
wherein
Rₑ and R_{f} are independently hydrogen or alkyl;
X is N, O or S; and
A₂ is
phenyl, naphthyl or biphenyl, each of which may be optionally substituted with one or more of C₁₋₄ alkyl, amino, aminoalkyl, halogen, hydroxy, —CF₃, alkoxy, aryloxy, arylalkoxy, —OCF₃, —COR_{g}, —COOR_{g}, —CONR_{g}Rₕ, —N(R₁)COR_{g}, —SO₂R_{g}, —SO₃R_{g} or —SO₂NR_{g}Rₕ; or
a 5- to 7-membered mono- or a 8- to 10-membered bicyclic heteroaromatic ring having from one to four heteroatoms selected from N, O or S, and may be optionally substituted with C₁₋₆ alkyl, amino, halogen, hydroxy, alkoxy, aryloxy, arylalkoxy, —CF₃, —OCF₃, —COR_{g}, —COOR_{g}, —CONR_{g}Rₕ, —NHCOR_{g}Rₕ, NHSO₂R_{g}, —SO₂R_{g}, —SO₃R_{g} or —SO₂NR_{g}Rₕ, wherein
R_{g} and Rₕ are independently hydrogen, alkyl, cycloalkyl, aryl, aralkyl, heteroaralkyl or heteroaryl.

5. A compound of claim 1, wherein 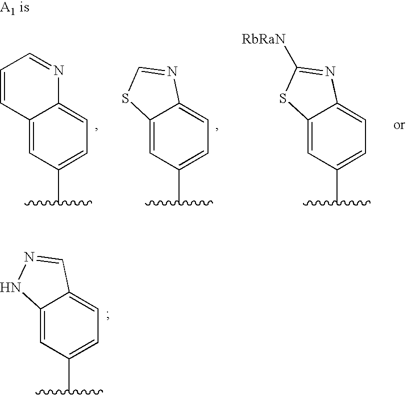
wherein Rₐ and R_{b} are independently —H, —C₁₋₆ alkyl, —CO₂-alkyl or —CO₂—CH₂CH₂NH₂;
R₁ is —H;
R₂ is
—H, —Me, -Et, 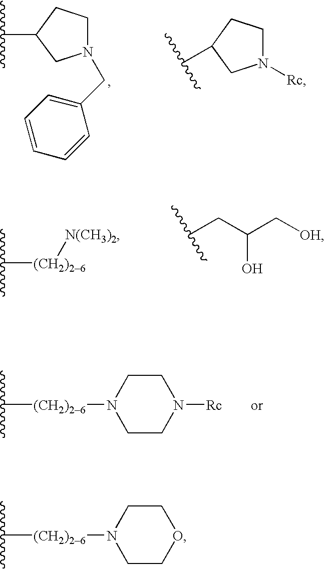
wherein R_{c} is alkyl;
R₃ is
—CH₂—, —CH₂CH₂—, —CH(CH₃)—, —C(CH₃)₂—, —CH(CH₂OH)— or —CH(CH₂CH₂COOH)—; and
A₂ is 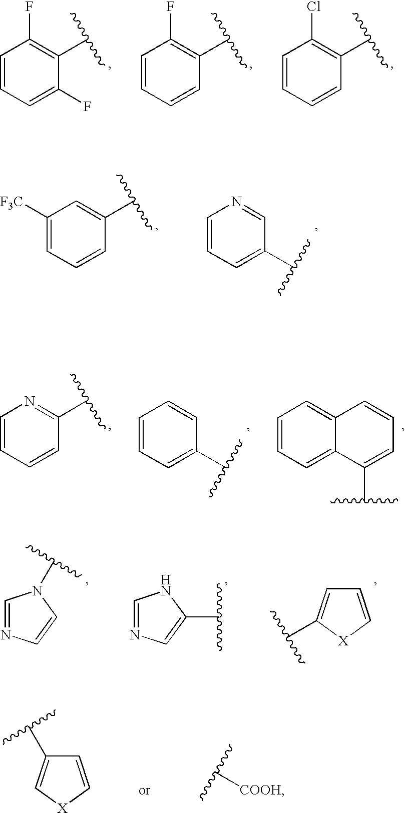
wherein X is O or S.

6. A compound of claim 1, which is one of
4-(Benzothiazol-6-ylamino)-6-(ethyl-benzylamino)-[1,3,5]triazin-2-ol;
4-(Benzothiazol-6-ylamino)-6-(methyl-benzylamino)-[1,3,5]triazin-2-ol;
4-(Benzothiazol-6-ylamino)-6-(benzylamino)-[1,3,5]triazin-2-ol;
(R)-4-(Benzothiazol-6-ylamino)-6-(1-phenylethylamino)-[1,3,5]triazin-2-ol;
(S)-4-(Benzothiazol-6-ylamino)-6-(1-phenylethylamino)-[1,3,5]triazin-2-ol;
(R)-4-(Benzothiazol-6-ylamino)-6-(methyl-1-phenylethylamino)-[1,3,5]triazin-2-ol;
(S)-4-(Benzothiazol-6-ylamino)-6-(methyl-1-phenylethylamino)-[1,3,5]triazin-2-ol;
(R)-4-(Benzothiazol-6-ylamino)-6-(ethyl-1-phenylethylamino)-[1,3,5]triazin-2-ol;
(S)-4-(Benzothiazol-6-ylamino)-6-(ethyl-1-phenylethylamino)-[1,3,5]triazin-2-ol;
4-(Benzothiazol-6-ylamino)-6-(1-methyl-1-phenylethylamino)-[1,3,5]triazin-2-ol;
4-(Benzothiazol-6-ylamino)-6-(2-phenylethylamino)-[1,3,5]triazin-2-ol;
4-(Benzothiazol-6-ylamino)-6-(methyl-2-phenylethylamino)-[1,3,5]triazin-2-ol;
4-(Benzothiazol-6-ylamino)-6-(ethyl-2-phenylethylamino)-[1,3,5]triazin-2-ol;
4-(Benzothiazol-6-ylamino)-6-(2-chloro-benzylamino)-[1,3,5]triazin-2-ol;
4-(Benzothiazol-6-ylamino)-6-(2-fluoro-benzylamino)-[1,3,5]triazin-2-ol;
4-(Benzothiazol-6-ylamino)-6-[(pyridin-3-ylmethyl)-amino]-[1,3,5]triazin-2-ol;
4-(Benzothiazol-6-ylamino)-6-(2,6-difluoro-benzylamino)-[1,3,5]triazin-2-ol;
4-(Benzothiazol-6-ylamino)-6-[methyl-(2-pyridin-2-yl-ethyl)amino]-[1,3,5]triazin-2-ol;
4-(Benzothiazol-6-ylamino)-6-[pyridin-2-ylmethyl)-amino]-[1,3,5]triazin-2-ol;
4-(Benzothiazol-6-ylamino)-6-[benzyl-(1-benzyl-pyrrolidin-3-yl)-amino]-[1,3,5]triazin-2-ol;
4-(Benzothiazol-6-ylamino)-6-(3-fluoro-benzylamino)-[1,3,5]triazin-2-ol;
4-(Benzothiazol-6-ylamino)-6-(2-chloro-6-methyl-benzylamino)-[1,3,5]triazin-2-ol;
4-(Benzothiazol-6-ylamino)-6-(N′-methyl-N′-phenyl-hydrazino)-[1,3,5]triazin-2-ol;
4-(benzothiazol-6-ylamino)-6-[(pyridin-4-ylmethyl)-amino]-[1,3,5]triazin-2-ol;
4-Benzothiazol-6-ylamino)-6-(2-pyridin-3-yl-ethylamino)-[1,3,5]triazin-2-ol;
4-Benzothiazol-6-ylamino)-6-(1-phenyl-propylamino)-[1,3,5]triazin-2-ol;
4-Benzothiazol-6-ylamino)-6-(2-pyridin-2-yl-ethylamino)-[1,3,5]triazin-2-ol;
4-(Benzothiazol-6-ylamino)-6-(1-naphthalen-1-yl-ethylamino)-[1,3,5]triazin-2-ol;
4-(Benzothiazol-6-ylamino)-6-(3-hydroxymethyl-phenylamino)-[1,3,5]triazin-2-ol;
4-(Benzothiazol-6-ylamino)-6-(quinolin-5-ylamino)-[1,3,5]triazin-2-ol;
4-(Benzothiazol-6-ylamino)-6-(4-hydroxy-naphthalen-1-ylamino)-[1,3,5]triazin-2-ol;
4-(Benzothiazol-6-ylamino)-6-(1H-indazol-6-ylamino)-[1,3,5]triazin-2-ol;
4-(Benzothiazol-6-ylamino)-6-[(1H-indazol-6-yl)-methylamino]-[1,3,5]triazin-2-ol;
4-(Benzothiazol-6-ylamino)-6-(1-methyl-1H-indazol-6-ylamino)-[1,3,5]triazin-2-ol;
4-(Benzothiazol-6-ylamino)-6-(6-hydroxy-naphthalen-1-ylamino)-[1,3,5]triazin-2-ol;
4-(Benzothiazol-6-ylamino)-6-(3-hydroxy-phenylamino)-[1,3,5]triazin-2-ol;
4-(Benzothiazol-6-ylamino)-6-[2-(2-hydroxyethyl)-phenylamino]-[1,3,5]triazin-2-ol;
4-(Benzothiazol-6-ylamino)-6-(5-thiophen-2-yl-2H-pyrazol-3-ylamino)-[1,3,5]triazin-2-ol;
4-(Benzothiazol-6-ylamino)-6-(2-phenyl-2H-pyrazol-3-ylamino)-[1,3,5]triazin-2-ol;
4-(Benzothiazol-6-ylamino)-6-(2,4-difluoro-benzylamino)-[1,3,5]triazin-2-ol;
4-(Benzothiazol-6-ylamino)-6-phenylamino-[1,3,5]triazin-2-ol;
4-(1H-Indazol-6-ylamino)-6-(1-methyl-1-phenylethylamino)-[1,3,5]triazin-2-ol;
4-(Benzothiazol-6-ylamino)-6-(2-hydroxy-1-phenylethylamino)-[1,3,5]triazin-2-ol;
4-(1H-Indazol-5-ylamino)-6-(1-methyl-1-phenylethylamino)-[1,3,5]triazin-2-ol;
4-(Benzothiazol-7-ylamino)-6-(1-methyl-1-phenylethylamino)-[1,3,5]triazin-2-ol;
4-(Benzothiazol-6-ylamino)-6-[(furan-2-yl-methyl)amino]-[1,3,5]triazin-2-ol;
4-(Benzothiazol-6-ylamino)-6-[(thiophen-2-yl-methyl)amino]-[1,3,5]triazin-2-ol;
4-(Benzothiazol-6-ylamino)-6-[(furan-3-ylmethyl)-amino-[1,3,5]triazin-2-ol;
4-(Benzothiazol-6-ylamino)-6-[(thiophen-3-yl-methyl)amino]-[1,3,5]triazin-2-ol;
4-(Benzothiazol-6-ylamino)-6-(benzyl-pyrrolidin-3-ylamino)-[1,3,5]triazin-2-ol;
3-{[4-(Benzothiazol-6-ylamino)-6-hydroxy-[1,3,5]triazin-2-yl]-benzylamino}-propane-1,2-diol;
4-(Benzothiazol-6-ylamino)-6-[benzyl-(3-morpholin-4-ylpropyl)-amino]-[1,3,5]triazin-2-ol;
4-(Benzothiazol-6-ylamino)-6-{benzyl-[3-(4-methyl-piperazin-1-yl)-propyl]-amino}-[1,3,5]triazin-2-ol;
4-(Benzothiazol-6-ylamino)-6-[benzyl-(3-dimethylamino-propyl)-amino]-[1,3,5]triazin-2-ol;
4-(Benzothiazol-6-ylamino)-6-[benzyl-(2-piperazin-1-ylethyl)-amino]-[1,3,5]triazin-2-ol;
4-(Benzothiazol-6-ylamino)-6-[benzyl-(2-morpholin-4-ylethyl)-amino]-[1,3,5]triazin-2-ol;
4-(Benzothiazol-6-ylamino)-6-[benzyl-(2-dimethylamino-ethyl)-amino]-[1,3,5]triazin-2-ol;
4-(2-Amino-benzothiazol-6-ylamino)-6-(1-methyl-1-phenylethylamino)-[1,3,5]triazin-2-ol;
4-(1-Methyl-1-phenylethylamino)-6-(quinolin-6-ylamino)-[1,3,5]triazin-2-ol;
4-(Quinolin-6-ylamino)-6-(N-ethylbenzylamino)-[1,3,5]triazin-2-ol;
4-(Quinolin-6-ylamino)-6-(N-methylbenzylamino)-[1,3,5]triazin-2-ol;
4-(Quinolin-6-ylamino)-6-(1-methyl-1-phenylethylamino)-[1,3,5]triazin-2-ol;
N-[4-(Benzothiazol-6-ylamino)-6-(1-methyl-1-phenylethylamino)-[1,3,5]triazin-2-yl]-hydroxylamine;
4-(Benzothiazol-6-ylamino)-6-[(4-fluoro-3-trifluoromethylbenzyl)amino]-[1,3,5]triazin-2-ol;
4-(Quinolin-6-ylamino)-6-[(4-fluoro-3-trifluoromethylbenzyl)amino]-[1,3,5]triazin-2-ol;
4-(Benzothiazol-6-ylamino)-6-(ethyl-(pyridin-2-ylmethyl)amino)-[1,3,5]triazin-2-ol;
4-(Benzothiazol-6-ylamino)-6-(N-benzylisopropylamino)-[1,3,5]triazin-2-ol;
4-(Benzothiazol-6-ylamino)-6-(ethyl-(2-fluorobenzyl)amino]-[1,3,5]trizin-2-ol;
4-(Benzothiazol-6-ylamino)-6-[benzyl-(2,2,2-trifluoroethyl)amino]-[1,3,5]triazin-2-ol;
3-[[4-(Benzothiazol-6-ylamino)-6-hydroxy-[1,3,5]triazin-2-yl]-(1-phenylethyl)amino]propane-1,2-diol;
4-(Benzothiazol-6-ylamino)-6-(ethyl-(pyridin-2-ylmethyl)amino)-[1,3,5]triazin-2-ol;
4-(Benzothiazol-6-ylamino)-6-(N-(2-fluorobenzyl)isopropylamino)-[1,3,5]triazin-2-ol;
4-(Benzothiazol-6-ylamino)-6-[ethyl-(1H-indazol-6-yl)amino]-[1,3,5]triazin-2-ol;
4-(Benzothiazol-6-ylamino)-6-{benzyl-[2-(3H-imidazol-4-yl)ethyl]amino}-[1,3,5]triazin-2-ol;
4-(Benzothiazol-6-ylamino)-6-{2-fluorobenzyl-[2-(3H-imidazol-4-yl)ethyl]amino}-[1,3,5]triazin-2-ol;
4-(Benzothiazol-6-ylamino)-6-[benzyl-(3-imidazol-1-yl-propyl)amino]-[1,3,5]triazin-2-ol;
4-{[4-(Benzothiazol-6-ylamino)-6-hydroxy-[1,3,5]triazin-2-yl]-benzylamino}butyric acid;
4-(Benzothiazol-6-ylamino)-6-{(2-piperazin-1-ylethyl)-quinolin-5-ylamino}-[1,3,5]triazin-2-ol; 4-(Benzothiazol-6-ylamino)-6-{benzyl-[2-(3H-imidazol-4-yl)ethyl]amino}-[1,3,5]triazin-2-ol;
4-(Benzothiazol-6-ylamino)-6-(N-benzylpropylamino)-[1,3,5]triazin-2-ol;
and pharmaceutically acceptable salts thereof.

7. A compound of claim 3, which is one of
4-(Benzothiazol-6-yl-amino)-6-(2-methyl-pyrrolidin-1-yl)-[1,3,5]triazin-2-ol;
4-(Benzothiazol-6-yl-amino)-6-(2-benzyl-pyrrolidin-1-yl)-[1,3,5]triazin-2-ol;
4-(Benzothiazol-6-yl-amino)-6-(2,6-dimethyl-piperidin-1-yl)-[1,3,5]triazin-2-ol;
4-(Benzothiazol-6-yl-amino)-6-(2,5-dimethyl-pyrrolidin-1-yl)-[1,3,5]triazin-2-ol;
4-(Benzothiazol-6-yl-amino)-6-(2-phenyl-pyrrolidin-1-yl)-[1,3,5]triazin-2-ol;
4-(Benzothiazol-6-yl-amino)-6-(3-phenyl-thiomorpholin-4-yl)-[1,3,5]triazin-2-ol;
4-(Benzothiazol-6-yl-amino)-6-(2-phenyl-thiomorpholin-4-yl)-[1,3,5]triazin-2-ol;
4-(Benzothiazol-6-yl-amino)-6-(thiomorpholin-4-yl)-[1,3,5]triazin-2-ol;
4-(Benzothiazol-6-yl-amino)-6-(3-methyl-piperidin-1-yl)-[1,3,5]triazin-2-ol;
4-(Benzothiazol-6-yl-amino)-6-(morpholin-4-yl)-[1,3,5]triazin-2-ol;
and pharmaceutically acceptable salts thereof.

8. A pharmaceutical composition, comprising a compound of any one of claims 1 to 4 and a pharmaceutically acceptable carrier.

9. A pharmaceutical composition, comprising a compound of claim 5 and a pharmaceutically acceptable carrier.

10. A pharmaceutical composition, comprising a compound of claim 6 or 7 and a pharmaceutically acceptable carrier.

11. A method of preparing the compounds of Formulae I and III where R is —OH, comprising the steps of:
a) displacing one of three displaceable groups at the 2-, 4- and 6-positions, respectively, of a 1,3,5-triazine ring with 4-methoxybenzyl alcohol to give a 2-(4-methoxybenzyloxy)-[1,3,5]triazine;
b) displacing the second displaceable group with a primary or secondary alkyl or aromatic amine (i) to give a 4-amino-2-(4-methoxybenzyloxy)-[1,3,5]triazine; and
c) displacing the third displaceable group with a primary or secondary alkyl or aromatic amine (ii) under microwave conditions with concomitant loss of the p-methoxybenzyl group to give a 4,6-diamino-(2-hydroxy)-[1,3,5]triazine.

12. A method of preparing the compounds of Formulae II and IV, comprising the steps of:
a) displacing one of three displaceable groups at the 2-, 4- and 6-positions, respectively, of a 1,3,5-triazine ring with 4-methoxybenzyl alcohol to give a 2-(4-methoxybenzyloxy)-[1,3,5]triazine;
b) displacing the second displaceable group with a primary or secondary alkyl or aromatic amine (i) to give a 4-amino-2-(4-methoxybenzyloxy)-[1,3,5]triazine;
c) displacing the third displaceable group with a primary or secondary alkyl or aromatic amine (ii) under microwave conditions with concomitant loss of the p-methoxybenzyl group to give a 4,6-diamino-(2-hydroxy)-[1,3,5]triazine; and
d) adding an acylating, sulfonylating or phosphorylating agent to the 4,6-diamino-(2-hydroxy)-[1,3,5]triazine to give a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

13. A method of claim 11 or 12, wherein the displaceable groups are chlorines.

14. A method of preparing the compounds of Formulae I and III where R is —OH, comprising the steps of:
aa) displacing one of three displaceable groups at the 2-, 4- and 6-positions, respectively, of a 1,3,5-triazine ring with a primary or secondary alkyl or aromatic amine (i) to give a 2-amino-[1,3,5]triazine;
bb) displacing the second displaceable group with a primary or secondary alkyl or aromatic amine (ii) to give a 2,4-diamino-[1,3,5]triazine; and
cc) displacing the third displaceable group with water under acidic conditions to give a 4,6-diamino-(2-hydroxy)-[1,3,5]triazine.

15. A method of preparing the compounds of Formulae I and III where R is —NHOH, comprising the steps of:
aa) displacing one of three displaceable groups at the 2-, 4- and 6-positions, respectively, of a 1,3,5-triazine ring with a primary or secondary alkyl or aromatic amine (i) to give a 2-amino-[1,3,5]triazine;
bb) displacing the second displaceable group with a primary or secondary alkyl or aromatic amine (ii) to give a 2,4-diamino-[1,3,5]triazine; and
cc) displacing the third displaceable group with hydroxylamine under acidic conditions to give a 4,6-diamino-([1,3,5]triazin-2-yl)-hydroxylamine.

16. A method of preparing the compounds of Formulae II and IV, comprising the steps of:
aa) displacing one of three displaceable groups at the 2-, 4- and 6-positions, respectively, of a 1,3,5-triazine ring with a primary or secondary alkyl or aromatic amine (i) to give a 2-amino-[1,3,5]triazine;
bb) displacing the second displaceable group with a primary or secondary alkyl or aromatic amine (ii) to give a 2,4-diamino-[1,3,5]triazine;
cc) displacing the third displaceable group with water under acidic conditions to give a 4,6-diamino-(2-hydroxy)-[1,3,5]triazine; and
dd) adding an acylating, sulfonylating or phosphorylating agent to the 4,6-diamino-(2-hydroxy)-[1,3,5]triazine to give a 4,6-diamino-(2-O-acyl)-[1,3,5]triazine, a 4,6-diamino-(2-O-sulfonyl)-[1,3,5]triazine or a 4,6-diamino-(2-O-phosphoryl)-[1,3,5]triazine, respectively.

17. A method for inhibiting protein tyrosine kinase activity, comprising contacting the kinase with an effective inhibitory amount of at least one compound of any one of claims 1 to 4.

18. A method for inhibiting protein tyrosine kinase activity, comprising contacting the kinase with an effective inhibitory amount of at least one compound of claim 5.

19. A method for inhibiting protein tyrosine kinase activity, comprising contacting the kinase with an effective inhibitory amount of at least one compound of claim 6 or 7.

20. A method for inhibiting protein tyrosine kinase activity in vitro, comprising contacting the kinase with at least one compound of any one of claims 1 to 4.

21. A method for inhibiting protein tyrosine kinase activity in vitro, comprising contacting the kinase with at least one compound of claim 5.

22. A method for inhibiting protein tyrosine kinase activity in vitro, comprising contacting the kinase with at least one compound of claim 6 or 7.

23. A method for inhibiting protein tyrosine kinase activity in cells, comprising contacting the kinase with at least one compound of any one of claims 1 to 4.

24. A method for inhibiting protein tyrosine kinase activity in cells, comprising contacting the kinase with at least one compound of claim 5.

25. A method for inhibiting protein tyrosine kinase activity in cells, comprising contacting the kinase with at least one compound of claim 6 or 7.

26. A method for inhibiting protein tyrosine kinase activity in a mammal, comprising administering to the mammal a therapeutically effective amount of at least one compound of any one of claims 1 to 4.

27. A method for inhibiting protein tyrosine kinase activity in a mammal, comprising administering to the mammal a therapeutically effective amount of at least one compound of claim 5.

28. A method for inhibiting protein tyrosine kinase activity in a mammal, comprising administering to the mammal a therapeutically effective amount of at least one compound of claim 6 or 7.

29. A method according to claim 17, wherein the protein tyrosine kinase is VEGFR-2 (KDR), c-fins, c-met or tie-2.

30. A method according to claim 26, wherein the protein tyrosine kinase is VEGFR-2 (KDR), c-fms, c-met or tie-2.

31. A method of treating cancer in a mammal, comprising administering to the mammal a therapeutically effective amount of at least one compound of any one of claims 1 to 4.

32. A method of treating cancer in a mammal, comprising administering to the mammal a therapeutically effective amount of at least one compound of claim 5.

33. A method of treating cancer in a mammal, comprising administering to the mammal a therapeutically effective amount of at least one compound of claim 6 or 7.

34. A method of treating vascular diseases in a mammal, comprising administering to the mammal a therapeutically effective amount of at least one compound of any one of claims 1 to 4.

35. A method of treating vascular diseases in a mammal, comprising administering to the mammal a therapeutically effective amount of at least one compound of claim 5.

36. A method of treating vascular diseases in a mammal, comprising administering to the mammal a therapeutically effective amount of at least one compound of claim 6 or 7.

37. A method of treating ocular diseases in a mammal, comprising administering to the mammal a therapeutically effective amount of at least one compound of any one of claims 1 to 4.

38. A method of treating ocular diseases in a mammal, comprising administering to the mammal a therapeutically effective amount of at least one compound of claim 5.

39. A method of treating ocular diseases in a mammal, comprising administering to the mammal a therapeutically effective amount of at least one compound of claim 6 or 7.

40. A method of treating restenosis in a mammal, comprising administering to the mammal a therapeutically effective amount of at least one compound of any one of claims 1 to 4.

41. A method of treating restenosis in a mammal, comprising administering to the mammal a therapeutically effective amount of at least one compound of claim 5.

42. A method of treating restenosis in a mammal, comprising administering to the mammal a therapeutically effective amount of at least one compound of claim 6 or 7.

43. A pharmaceutical dosage form comprising a pharmaceutically acceptable carrier and from about 0.5 mg to about 10 g of at least one compound of any one of claims 1 to 7.

44. A dosage form according to claim 43 adapted for parenteral or oral administration.
